(19) United States
(12) Patent Application Publication
(10) Pub. No.: US 2010/0000881 A1
(43) Pub. Date: Jan. 7, 2010

(54) Electrochemical detection of nucleic acid hybridization

(75) Inventors: Stefan Franzen, Apex, NC (US); Daniel Feldheim, Cary, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: North Carolina State University

(21) Appl. No.: 10/978,678

(22) Filed: Nov. 1, 2004

Related U.S. Application Data
(60) Provisional application No. 60/515,961, filed on Oct. 30, 2003.

(51) Int. Cl. C12Q 1/68; G01N 27/26

(52) U.S. Cl. 205/780.5; 435/6

(57) ABSTRACT

A nucleic acid hybridization detection assay is carried out at an electrode. A solid electrode, such as an indium tin oxide electrode, is modified by capture probes comprising single-stranded oligonucleotides immobilized to the surface of the electrode. In some embodiments using sandwich assay methodology, the capture probes hybridize complementary target nucleic acid sequences, which in turn are bound to detection probes comprising nanoparticle-oligonucleotide conjugates comprising target-complementary oligonucleotides. In some embodiments, detection probes comprise nanoparticles attached to molecules comprising one partner of a ligand-binding pair (e.g., streptavidin), while target sequences comprise the other partner of the ligand-binding pair (e.g., biotin). When the assay is carried out in the presence of a redox mediator, redox reactions catalyzed, and/or facilitated and/or enhanced by the presence of nanoparticles generate electrons that are transferred to the electrode, resulting in a detectable electrical signal.

### RELATED APPLICATIONS

This application claims the benefit of U.S. Provisional Patent Application Ser. No. 60/515,961 filed Oct. 30, 2003; the disclosure of which is incorporated herein by reference in its entirety.

### TECHNICAL FIELD

The described methods relate to electrochemical detection of biological molecules such as nucleic acids. In particular, nanoparticles and target-specific probes are utilized in catalytic and electrochemical methods for detecting nucleic acid hybridization.

### BACKGROUND

The detection of specific nucleic acid sequences in biological samples provides a basis for myriad practical and research techniques, including gene identification, mutation detection, gene expression profiling, and DNA sequencing. Diagnostic and forensic applications are but two areas in which nucleic acid detection techniques find widespread use.

Particular nucleic acid sequences are usually detected by one or more nucleic acid hybridization assays, in which the presence of a target sequence in a biological sample is determined by hybridizing a probe sequence designed to specifically bind the target with heterogeneous nucleic acids in the sample. The presence of the target is usually indicated by the detection of a chemical, enzymatic, magnetic or spectroscopic label that is directly or indirectly attached to either the probe or the target sequence. Such hybridization assays are increasingly being combined with parallel, high-throughput microarray technology, in which thousands of hybridization assays are carried out simultaneously on a solid substrate (e.g., a “chip”). Microarray technologies are highly amenable to automation and facilitate the screening of, for example, one biological sample against a large number of probes in a brief time period.

The utility, versatility and diagnostic value of any particular system for detecting nucleic acid sequences of interest can be limited. For example, fluorescent labeling and detection methodologies are generally not sufficiently sensitive to single-base mismatches in surface-bound hybridization duplexes. Additionally, fluorescence-based techniques require extensive sample preparation, as well as the use of unwieldy apparatus such as confocal microscopes. Moreover, many commonly used labeling and detection techniques also require the use of costly and time-consuming nucleic acid amplification techniques. Sensitive methods that are able to differentially detect target nucleic acids thus remain in demand.

### SUMMARY

Described herein are sensitive electrochemical methods for detecting nucleic acid sequences and nucleic acid hybridization events.

In some embodiments provided are methods of detecting a target nucleic acid sequence, comprising providing a hybridization complex comprising (a) a capture probe that is attached to an electrode and (b) a target nucleic acid sequence that is hybridized to the capture probe, wherein the target nucleic acid sequence additionally comprises at least one nanoparticle attached to the target nucleic acid sequence, such that the complex is in contact with a redox solution comprising a redox mediator and an electrolyte, and detecting an electrical signal in the electrode, whereby detection of an increased electrical signal relative to a signal that would be detected in the absence of said complex indicates the presence or amount of target nucleic acid sequence hybridized to the electrode.

In some embodiments, a target nucleic acid sequence hybridizes a capture oligonucleotide probe that is attached to an electrode. The target sequence is then hybridized with a detection probe comprising a nanoparticle, thus forming a capture probe-target sequence-detection probe hybridization complex at the surface of the electrode.

Contacting the electrode with a redox mediator in solution results in one or more redox reactions that elicit the transfer of electrons to the electrode. The redox reaction(s) proceeds in the presence of the nanoparticle attached to the electrode via the hybridization complex. In some embodiments, the nanoparticle is involved in electron transfer from the redox mediator in solution to the electrode. In some embodiments, the nanoparticle catalyzes the redox reaction. In some embodiments, redox active moieties attached to the nanoparticle are oxidized by the electrode, while other redox active moieties (e.g., electron donors) in solution reduce or re-reduce the nanoparticle-attached redox active moieties.

The transfer of electrons to the electrode driven by the redox reactions generates a detectable electrical signal (e.g., current or potential) in the electrode. In certain embodiments, the presence of an electron donor such as EDTA in the redox solution can advantageously amplify the detectable electrical signal. The detected electrical signal provides a measure of nucleic acid hybridization at the surface of the electrode, which can be correlated with the concentration of target nucleic acid present in the sample.

In some embodiments, target sequences and capture probes comprise single-stranded nucleic acid regions, while detection probes comprise a nanoparticle-oligonucleotide conjugate. In some embodiments, the detection probe comprises a nanoparticle attached to at least one partner of a ligand-binding pair (for example, streptavidin), while the target nucleic acid comprises the other, corresponding binding partner of the ligand-binding pair (for example, biotin). In some embodiments, the target sequence is tagged with biotin moieties during an amplification reaction in which single stranded nucleic acid (e.g., mRNA) is used as a template, and biotin-tagged nucleotides are enzymatically incorporated into a complementary cDNA strand (e.g., by reverse transcriptase).

In some embodiments, the electrode and nanoparticles used in the described methods comprise different materials that are or comprise one or more metals and/or metal oxides. In a particular embodiment, the electrode comprises indium tin oxide, and the nanoparticle comprises a metal selected from the group consisting of gold (Au), silver (Ag) and platinum (Pt).

In some embodiments, the electrical signal produced in the electrode is electrical current, and is detected using voltammetry. In another embodiment, the electrical signal produced in the electrode is detected using chronoamperometry.

It is therefore an object of the present invention to provide a method of detecting nucleic acid hybridization. This object is achieved in whole or in part by the methods described in more detail below.

### BRIEF DESCRIPTION OF THE DRAWINGS

FIGS. 1A and 1B are schematic diagrams illustrating an electrochemical detection strategy. FIG. 1A is an illustration of an indium tin oxide electrode, to which a plurality of capture probes are attached. An indium tin oxide electrode is shown for the purposes of illustration only, and not for the purposes of limitation. FIG. 1B illustrates an embodiment of the present methods in which a target cDNA is hybridized to capture probes attached to the electrode surface shown in FIG. 1A. A detection probe, comprising a gold nanoparticle and an oligonucleotide is hybridized to the target cDNA; accordingly, a hybridization complex comprising a capture probe, a target sequence and a detection probe is illustrated. The electrode surface, with a hybridization complex attached, is in the presence of an electrolyte solution comprising a redox molecule. A gold nanoparticle is shown for the purposes of illustration only, and not for the purposes of limitation.

FIG. 2 is a schematic diagram illustrating a mechanism by which electrochemical detection of nucleic acid hybridization is facilitated. An indium tin oxide (ITO) electrode is illustrated as having capture oligonucleotide probes attached thereto. An indium tin oxide electrode is shown for the purposes of illustration only, and not for the purposes of limitation. In the absence of a nanoparticle, redox molecules in solution undergo a redox reaction that is kinetically slow (i.e., very little electron transfer to the electrode occurs), thus generating a low current in the electrode. This situation is illustrated in the left-hand side of the Figure. In the right-hand side of the Figure, target cDNA molecules are brought into contact with the electrode in the presence of nanoparticles (shown in the Figure with single-stranded DNA probes attached thereto) and a redox solution comprising redox mediators. A hybridization complex is formed between the capture probes, the target cDNA and the oligonucleotides attached to the nanoparticle, in effect attaching the nanoparticles to the electrode surface. A redox reaction elicits a flow of electrons (i.e., electron transfer) to the surface of the electrode, and thus generates a high current in the electrode. In some embodiments, the nanoparticle is involved in electron transfer from the redox mediator in solution to the electrode surface. In certain embodiments, the nanoparticle functions as a catalyst for the redox reaction. The difference between the high electrical current generated by the nanoparticle-modified electrode and the low electrical current generated by the complex-free electrode (i.e., no attached nanoparticles) provides a measure of nucleic hybridization at the electrode surface.

FIGS. 3A, 3B and 3C, taken together, further illustrate experimentally observed electrochemical mechanisms useful for the detection of nucleic acids. The left-hand side of FIG. 3A essentially corresponds to the reaction scenario illustrated in the left-hand side of FIG. 2; that is, in the absence of nanoparticles, electron transfer (ET) from a redox species in solution to the electrode surface is slow. FIG. 3B is a representative illustration of a current trace that is generated under this scenario, where current detected in the ITO electrode as a result of any electron transfer thereto is plotted as a function of a sweep in potential between about 0 and about 1.2 volts. The right-hand side of FIG. 3A essentially corresponds to the reaction scenario illustrated in the right-hand side of FIG. 2; that is, in the presence of nanoparticles attached to the electrode surface by means of nucleic acid hybridization, electron transfer (in this case, from the redox species in solution to the electrode surface) is significantly faster than the situation illustrated in the left-hand side of FIG. 3A. FIG. 3C is a representative illustration of a current trace that is generated under the fast-ET scenario, where current in the electrode is plotted as a function of a potential sweep between about 0 and about 1.2 V. A significant difference in the traces of FIGS. 3B and 3C is observed, with a marked peak (anodic current spike) being observed as the applied potential nears about 0.9-1.0 V in the sweep cycle. An indium tin oxide electrode is shown for the purposes of illustration only, and not for the purposes of limitation. A gold nanoparticle is shown for the purposes of illustration only, and not for the purposes of limitation.

FIG. 4 is a schematic diagram illustrating a method by which nucleic acid molecules can be attached to a nanoparticle.

FIG. 5 illustrates the formation of an amide bond by the activation of the carboxylic acid on a monolayer of 12-phosphonododecanoic acid on ITO by 1-ethyl-3-(3-dimethylaminopropyl)carbodiimide hydrochloride (EDC) with 5′ modified C₃H₂ single-stranded (ss) DNA. ITO is shown for the purposes of illustration only, and not for the purposes of limitation.

FIG. 6 is an x-ray photoelectron spectra (XPS) of ln 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 7 is an XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

FIG. 8 is an XPS N 1 s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

FIG. 9 is an XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

FIG. 10 is a graph comparing the cyclic voltammogram trace of gold nanoparticles hybridized onto ITO electrodes when the electrode solution comprises an electrolyte solution without EDTA (KP buffer only, upper trace/small current peak observed) and with EDTA (KP buffer/EDTA, lower trace/large current peak observed). In the absence of EDTA, redox waves associated with gold oxide formation and re-reduction are observed. When EDTA is added to the redox solution, the gold oxide wave increases in magnitude. The presence of EDTA thus causes a significant enhancement in the signal from surface-bound gold nanoparticles.

FIG. 11 is a graph of current as a function of sweeping potential, and illustrates the detection of gold particles bound to ITO electrodes. In the experiments illustrated in FIG. 11, 1 picomole (pmole) of 10 nanometer (nm) gold particles were capped with citrate and attached to the surface of an ITO electrode using the aminosilane attachment chemistry described in K. C. Grabar et al., J. Am. Chem. Soc. (1996) 118, 1148. Electrochemistry was carried out in a solution of 100 mM KP buffer/50 mM EDTA, pH. 7.3, and with a potential sweep rate of 100 mV/s. A detectable peak is observed at about 0.9 V.

FIG. 12 is a comparison of cyclic voltammetry traces between gold particles bound to indium tin oxide electrodes when complementary single stranded DNA is attached to each (solid line) and when non-complementary single stranded DNA is attached to each (broken line). The cyclic voltammagram was obtained in 100 mM potassium phosphate, 50 mM EDTA, pH 7.3; 100 mV/s scan rate; Indium-tin oxide substrate cleaned for 15 minutes by UV-ozonolysis; 500 picomolar target oligonucleotide in solution labeled with 10 nm gold particles stabilized with BSPP.

FIG. 13 is an illustration of the limits of detection of methods of the present invention. The present electrochemical methods are able to detect (i.e., distinguish over background) hybridization of nucleic acids at electrode surfaces in concentrations as low as about 10 μM. The cyclic voltammagram obtained in 100 mM FeCl₂; 100 mV/s scan rate; Indium-tin oxide substrate cleaned for 15 minutes by UV-ozonolysis; hybridization for 19 hours at 37° C. while gently stirring the solution; 10 picomolar target oligonucleotide in solution labeled with 10 nm gold particles stabilized with BSPP.

FIGS. 14A and 14B, taken together, provide a graphical comparison of a known method of incorporating a fluorescent label into a target nucleic acid (FIG. 14A), and a presently-described method of incorporating one partner of ligand-binding pair (e.g., biotin) into a target nucleic acid, which ligand-binding pair partner can then be used to bind a nanoparticle to which is attached the other member of the ligand-binding pair (e.g., streptavidin) (FIG. 14B).

### DETAILED DESCRIPTION

The presently disclosed subject matter will be now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs. All publications, patent applications, patents, and other references mentioned herein are incorporated by reference in their entirety. Throughout the specification and claims, a given chemical formula or name shall encompass all optical and stereoisomers as well as racemic mixtures where such isomers and mixtures exist.

### I. General Overview of Methods

The methods described herein can be used for detecting nucleic acid hybridization events. More particularly, the present methods are useful for detecting specific target nucleic acid sequences in a heterogeneous sample.

In some embodiments, a method of detecting a target nucleic acid sequence comprises providing a hybridization complex comprising (a) a capture probe that is attached to an electrode and (b) a target nucleic acid sequence that is hybridized to the capture probe, wherein the target nucleic acid sequence additionally comprises at least one nanoparticle attached to the target nucleic acid sequence, such that the complex is in contact with a redox solution comprising a redox mediator and an electrolyte, and detecting an electrical signal in the electrode, whereby detection of an increased electrical signal relative to a signal that would be detected in the absence of the complex indicates the presence or amount of target nucleic acid sequence hybridized to the electrode. The electrical signal that is detected can be a non-photo-dependent or non-photogenerated electrical signal.

In some embodiments the presently disclosed methods are free of (i.e. do not involve) the use of a target analyte attached to a conductive support and/or a nanoparticle comprising a photoelectrochemically active moiety such as a ruthenium complex (e.g., ruthenium tris-bipyridine and related adducts that have long-lived excited states). In some embodiments the presently disclosed methods are free of a nanoparticle comprising a photoelectrochemically active moiety such as a ruthenium complex (e.g., ruthenium tris-bipyridine and related adducts that have long-lived excited states). In some embodiments the presently disclosed methods are free of a photoelectrochemically active moiety that performs a “dye” or label function in the practice of these methods. In some embodiments the presently disclosed subject matter is free of exposing the photoelectrochemically active moiety to light (e.g. a laser).

In some embodiments, a nucleic acid hybridization detection assay is carried out at a solid electrode surface. A solid electrode, such as an indium tin oxide electrode, is modified by single-stranded capture oligonucleotide probes that are immobilized to the surface of the electrode. The capture probes hybridize with complementary target nucleic acid sequences, which are in turn hybridized by a detection probe comprising a nanoparticle. Thus, the target sequence forms part of a hybridization complex comprising a capture probe, a target sequence, and a detection probe.

As used herein, the terms “complex”, “duplex,” and “hybridization complex” are used interchangeably, and mean a structure formed of at least two different members. Hybridization complexes can comprise two or more DNA sequences, RNA sequences or combinations thereof. Complexes, in general, form via hybridization of complementary strands of nucleic acids by Watson Crick or Hoogsteen base-pairing, or by the specific binding of ligand-binding pairs such as streptavidin and biotin. A member of a hybridization complex can itself comprise one, two or more members. Thus, a complex can comprise a structure comprising two members, one or both of which can itself be a complex. For example, one member of a complex can comprise a single stranded nucleic acid sequence (immobilized or in solution), and the second member of the complex can comprise a nucleic acid double stranded complex (immobilized or in solution), effectively making the complex a triplex structure.

The term “target sequence,” as used herein, means a nucleic acid sequence on a single strand of nucleic acid. A target sequence may accordingly be a single-stranded segment of a target nucleic acid. If the target nucleic acid is single-stranded, the target sequence can be identical to the target nucleic acid, or may comprise a portion or sub-sequence of the target nucleic acid. If the target nucleic acid is double-stranded DNA, the target sequence may be identical to or comprise a sub-sequence of the coding strand, or may be identical to or comprise a sub-sequence of the anti-parallel, complementary, non-coding strand. As described in further detail below, target sequences may optionally comprise moieties such as labels or tags that facilitate specific binding to a detection probe comprising a nanoparticle.

A “capture probe,” as used herein, is an oligonucleotide that binds (i.e., hybridizes) to a target nucleic acid sequence, and which is used to probe for the presence of the target sequence. The capture probe enables the attachment of a target nucleic acid to a solid electrode for the purposes of detection. A “detection probe,” as used herein, comprises a nanoparticle. In some embodiments, a detection probe comprises a nanoparticle-oligonucleotide conjugate. Thus, each probe can comprise an oligonucleotide sequence attached to either a particle or a solid surface. In general, a capture probe is bound to an electrode surface, while a detection probe comprises an oligonucleotide attached to a nanoparticle. In some embodiments, a detection probe comprises one partner of a ligand-binding pair (e.g., streptavidin) instead of an oligonucleotide. In these embodiments, a detection probe thus comprises a nanoparticle attached to one partner of a ligand-binding pair.

Nanoparticles and electrodes of the present invention may be fabricated from a broad range of materials, with one limitation being that the nanoparticle material and the electrode material are not identical. Moreover, the electrode generally comprises a conductive material. Accordingly, the nanoparticles and electrodes used in the present methods typically comprise metal or metal oxide materials.

In some embodiments, a capture oligonucleotide probe hybridizes a first domain of the target sequence, while an oligonucleotide component of a detection probe hybridizes a second domain of the target sequence to form a hybridization complex. In other embodiments, the detection probe can bind to the same domain as the capture probe, forming a triplex.

Detection of the hybridization complex is facilitated by contacting the electrode surface with a redox solution comprising redox mediator in an electrolyte solution. Contact with the redox solution can occur either concurrently with or subsequent to the formation of the hybridization complexes. The redox mediator in solution participates in a redox reaction which elicits electron transfer to the electrode surface, thus creating a detectable electrical current in the electrode. In some embodiments, the nanoparticle attached to the electrode surface catalyzes the redox reaction.

In some embodiments, an electrochemically active moiety such as EDTA is present in the redox solution. In some embodiments, the oxidation of electron-donating compounds such as EDTA is catalyzed by the presence of the nanoparticle, thus amplifying the electrical signal detected in the electrode. The nanoparticle itself can also function as a “redox-active signal”. For example, a single gold nanoparticle comprises tens of thousands of gold atoms that can be oxidized to Au³⁺ ions. This oxidation reaction can be detected electrochemically, and also serves to amplify the detected electrical signal.

Comparing the difference between electrical signal (e.g., current) generated in the electrode by the electron transfer and the electrical signal generated by a complex-free electrode (e.g., an electrode unmodified by nanoparticles) provides a measure of nucleic acid hybridization at the electrode surface. Alternatively, comparing the difference in potential between the nanoparticle-modified electrode and the complex-free electrode provides a measure of nucleic acid hybridization at the electrode surface. This measured signal can be correlated to the concentration of target nucleic acid in the sample.

In some embodiments disclosed are sandwich assay methodology, nucleic acid microarray technology and catalytic electrochemical detection techniques. In accordance with experiments described herein, detection sensitivities on the order of about 10 μM or better have been obtained.

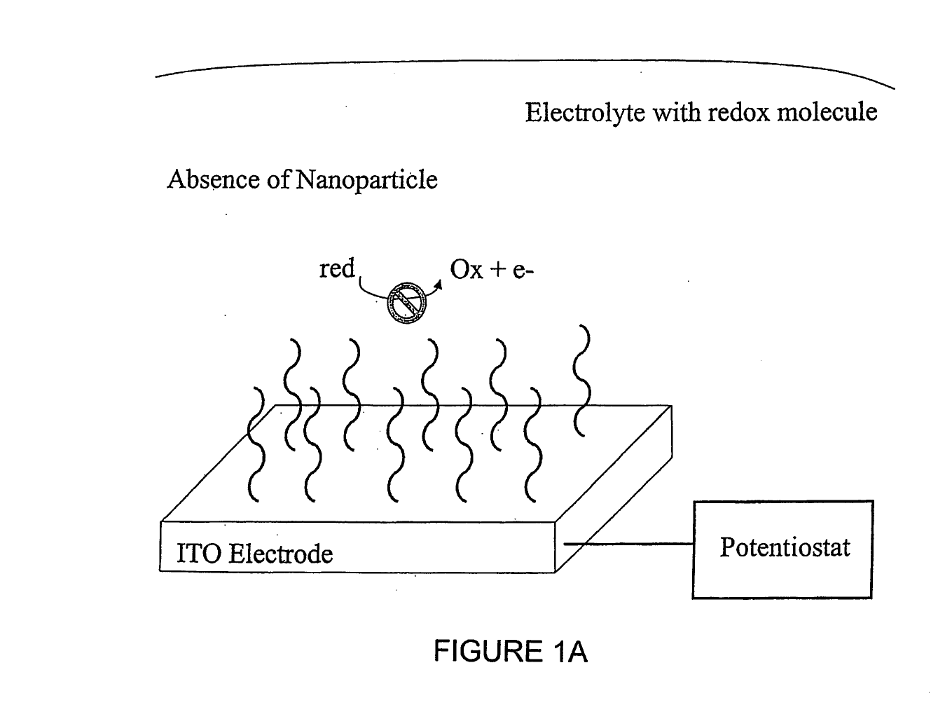

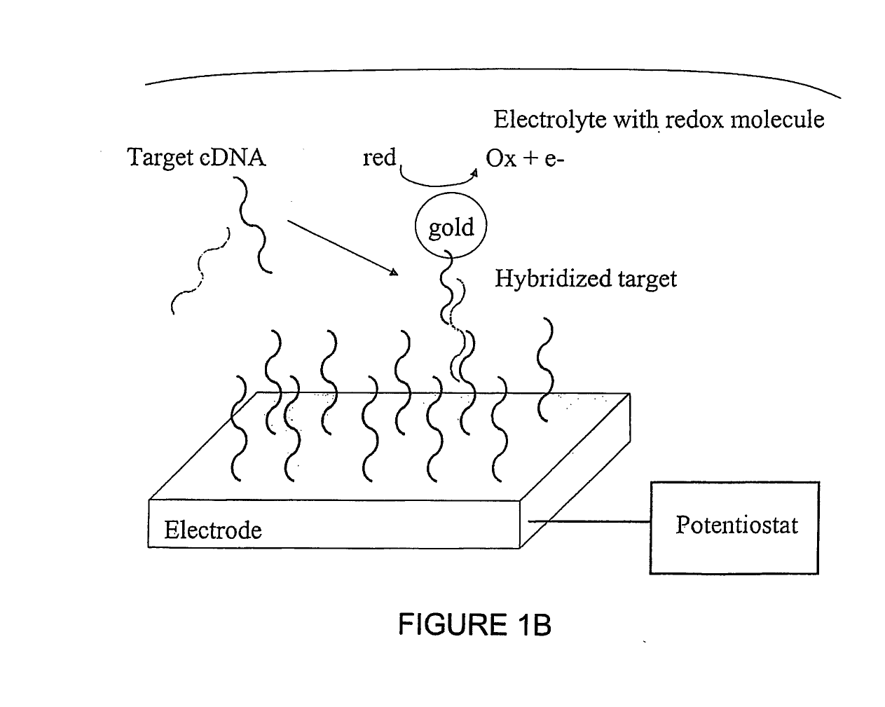

FIGS. 1A and 1B, taken together, provide a graphical illustration of the electrochemical methods described herein. FIG. 1A is an illustration of an electrode (e.g. an ITO electrode) to which a plurality of capture probes have been attached. Neither target sequences nor detection probes comprising nanoparticles are present, although the electrode is shown as being in the presence of a redox solution. A redox reaction releasing an electron is not catalyzed in this scenario, as illustrated in the Figure.

FIG. 1B illustrates a scenario in which a target cDNA is hybridized to capture probes attached to the electrode (e.g. an ITO electrode) surface shown in FIG. 1A. A detection probe comprising a gold nanoparticle and an oligonucleotide is hybridized to the target cDNA; accordingly, a hybridization complex comprising a capture probe, target sequence and detection probe is formed. The electrode surface, with the hybridization complex attached, is in the presence of an electrolyte solution comprising a redox mediator. The resulting redox reaction generates an electron, and the resulting electric current produced by the electron transfer to the electrode is measured with reference to controlled potential, shown as being controlled by a potentiostat in the Figure.

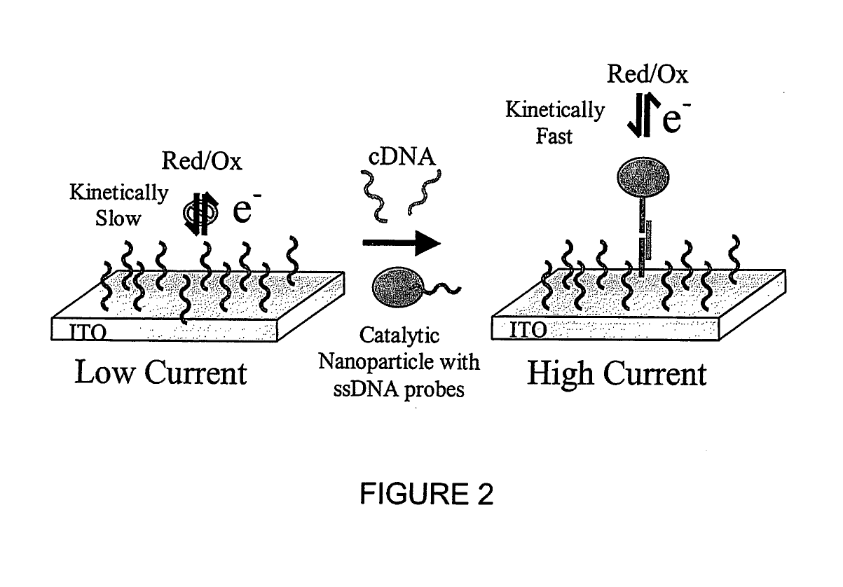

FIG. 2 further illustrates a mechanism by which the detection of nucleic acid hybridization is facilitated. An electrode (e.g. an ITO electrode) is illustrated as having capture oligonucleotide probes attached thereto. In the absence of a nanoparticle, redox molecules in solution undergo a redox reaction that is kinetically slow (i.e., very little electron transfer from the solution to the electrode occurs), thus generating a low current in the electrode. This situation is illustrated in the left-hand side of the Figure. In the right-hand side of the picture, target cDNA molecules are brought into contact with the electrode in the presence of nanoparticles (shown in the Figure with single-stranded DNA probes attached thereto) and a redox solution comprising a redox mediator. A hybridization complex is formed between the capture probes, the target cDNA and the oligonucleotides attached to the nanoparticle, in effect attaching the nanoparticles to the electrode surface. A redox reaction elicits a flow of electrons (i.e., electron transfer) to the surface of the electrode and thus generates a high current in the electrode. In some embodiments, the nanoparticle functions as a “bridge” for electron transfer from the redox mediator in solution to the electrode surface. In some embodiments, the nanoparticle functions as a catalyst for the redox reaction. The difference between the high electrical current generated by the nanoparticle-modified electrode and the low electrical current generated by the complex-free electrode (i.e., no attached nanoparticles) is a measure of nucleic hybridization at the electrode surface.

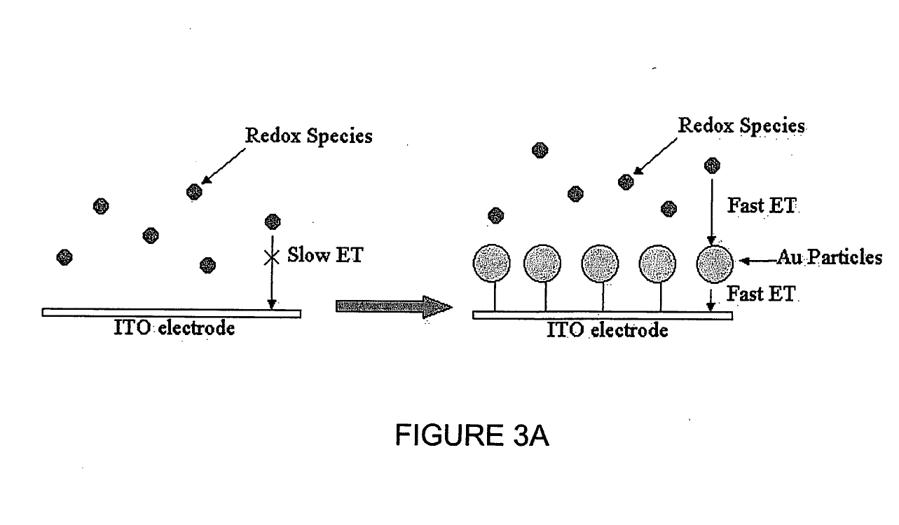

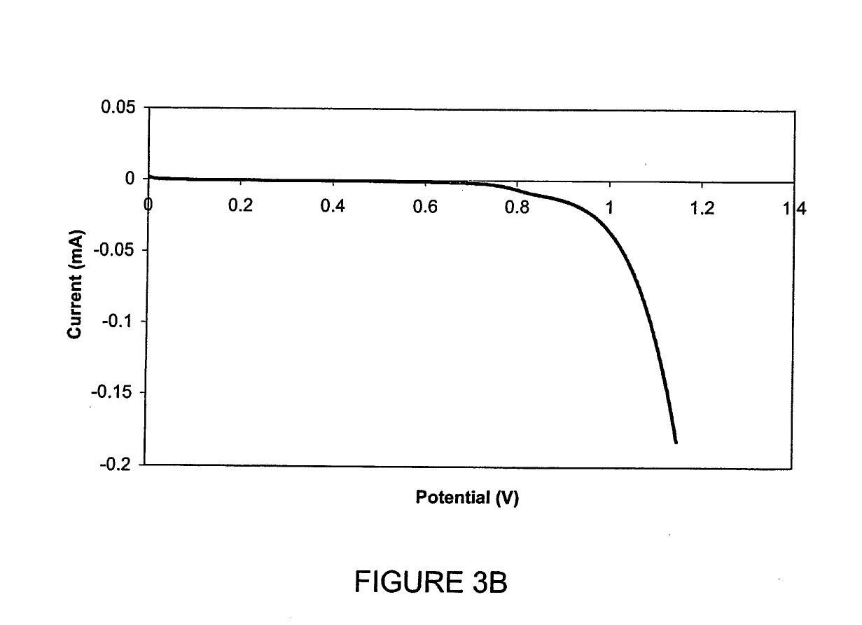

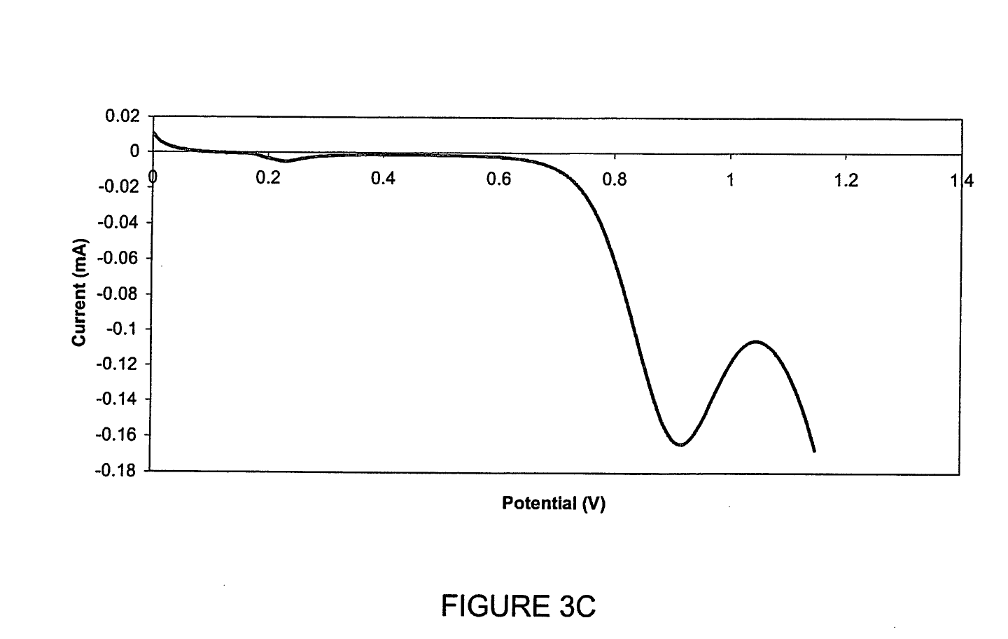

FIGS. 3A, 3B and 3C, taken together, further elucidate electrochemical mechanisms useful for the detection of nucleic acids. The left-hand side of FIG. 3A essentially corresponds to the reaction scenario illustrated in the left-hand side of FIG. 2; that is, in the absence of nanoparticles, electron transfer (ET) from a redox species in solution to the electrode surface is slow. FIG. 3B is a representative illustration of a current trace that is generated under this scenario, where current detected in the ITO electrode as a result of any electron transfer thereto is plotted as a function of a sweep in potential between about 0 and about 1.2 volts. The right-hand side of FIG. 3A essentially corresponds to the reaction scenario illustrated in the right-hand side of FIG. 2; that is, in the presence of nanoparticles attached to the electrode surface by means of nucleic acid hybridization, electron transfer (in this case, from the redox species in solution to the electrode surface) is significantly faster than the situation illustrated in the left-hand side of FIG. 3A. FIG. 3C is a representative illustration of a current trace that is generated under the fast-ET scenario, where current in the electrode is plotted as a function of a potential sweep between about 0 and about 1.2 V. A significant difference in the traces of FIGS. 3B and 3C is observed, with a marked peak (anodic current spike) being observed as the applied potential nears about 0.9-1.0 V in the sweep cycle.

### II. Nucleic Acid Sequences

The methods described herein are useful for the detection of target nucleic acid sequences and nucleic acid hybridization events. Probes useful in the detection of target sequences and nucleic acid hybridization events comprise nucleic acids in the form of oligonucleotides.

As used herein, the terms “nucleic acid,” “nucleic acid sequence,” “nucleic acid molecule,” and grammatical equivalents mean at least two nucleotides covalently linked together. Nucleic acids may be single-stranded or double-stranded, as specified, or contain portions of both double-stranded or single-stranded sequence. Nucleic acids can comprise any combination of deoxyribo- and ribonucleotides, and any combination of bases, including uracil, adenine, thymine, cytosine, guanine, inosine, xathanine hypoxathanine, isocytosine, isoguanine, etc. Deoxyribonucleic acids (DNA) can comprise genomic DNA, cDNA derived from ribonucleic acid, DNA from an organelle (e.g., mitochondrial DNA or chloroplast DNA), synthesized DNA (e.g., oligonucleotides), or combinations thereof. Ribonucleic acids (RNA) can comprise genomic RNA (e.g., viral genomic RNA), messenger RNA (mRNA), ribosomal RNA (rRNA), transfer RNA (tRNA), or combinations thereof.

A nucleic acid of the present invention will generally contain phosphodiester bonds, although in some cases, as outlined below, nucleic acid analogs are included that may have alternate backbones, comprising, for example, phosphoramide, phosphorodithioate, methylphosphoroamidite linkages, and peptide nucleic acid backbones and linkages. Other analog nucleic acids include those with positively-charged backbones, non-ionic backbones and nonribose backbones. Nucleic acids containing one or more carbocyclic sugars are also included within the definition of nucleic acids. Mixtures of naturally occurring nucleic acids and analogs can be used. Alternatively or in addition, mixtures or chimeras of different nucleic acid analogs, and mixtures of naturally occurring nucleic acids and analogs can be used.

Peptide nucleic acids (PNA) are specifically included in the definition of nucleic acids, as used herein. PNAs are DNA analogs wherein the backbone of the analog (for example, a sugar backbone in DNA) is a pseudopeptide. A PNA backbone can comprise, for example, a sequence of repeated N-(2-amino-ethyl)-glycine units. A peptide nucleic acid analog reacts as DNA would react in a given environment, and can bind complementary nucleic acid sequences and various proteins. Peptide nucleic acid analogs offer the potential advantage over unmodified DNA of the formation of stronger bonds, due to the neutrally charged peptide backbone of the analogs, and can impart a higher degree of specificity than is achievable by unmodified DNA. These backbones are substantially non-ionic under neutral conditions, in contrast to the highly charged phosphodiester backbone of naturally occurring nucleic acids.

Nucleic acids can also comprise “locked nucleic acids”, also known as LNAs (e.g., WO 98/39352).

When used as oligonucleotide probes, as defined herein, nucleic acids can be analytically pure, as determined by spectrophotometric measurements or by visual inspection following electrophoretic resolution. In some embodiments, nucleic acids that are to be amplified can be analytically pure, although purity is not a requirement. In some embodiments, nucleic acid samples are free of contaminants such as polysaccharides, proteins and inhibitors of enzyme reactions. When an RNA sample is intended for use as probe or target sequence, it is preferably free of DNAase and RNAase. Contaminants and inhibitors can be removed or substantially eliminated using resins for DNA extraction or by standard phenol extraction and ethanol precipitation, as is taught in the art.

A. Target Nucleic Acids and Sequences

A target sequence can be selected on the basis of the context in which the present methods are employed. Target sequences can vary widely. For example, desirable target sequences include, but are not limited, to characteristic or unique nucleic acid sequences found in various microbes or mutated DNA that can be used in the diagnosis of diseases, in environmental bioremediation, in the determination of genetic disorders, and in genetic epidemiology. Functional equivalents of known sequences can also be used as target sequences.

The target sequence may be a portion of a gene, a regulatory sequence, genomic DNA, cDNA, RNA including mRNA and rRNA, or others. The target sequence can be a target sequence from a biological sample, as discussed herein, or can be a secondary target such as a product of an amplification reaction. The target sequence can take many forms. For example, a target may be contained within a larger nucleic acid sequence, i.e. all or part of a gene or mRNA, a restriction fragment of a plasmid or genomic DNA, among others. Target nucleic acids can be excised from a larger nucleic acid sample using restriction endonucleases, which sever nucleic acid sequences at known points in a nucleic acid sequence. Excised nucleic acid sequences can be isolated and purified by employing standard techniques. Target sequences can also be prepared by reverse transcription processes. See, e.g., Sambrook et al., Molecular Cloning: A Laboratory Manual (Cold Spring Harbor, N.Y. (1992)).

A target sequence can comprise one or more different target domains. A target domain is a contiguous, partial sequence (i.e., a sub-sequence) of the entire target sequence, and may be any nucleotide length that is shorter than the entire target sequence. In some embodiments, a first target domain of a target sequence hybridizes a capture probe, while a second and different target domain hybridizes an oligonucleotide component of a detection probe. Target domains may be adjacent or separated, as indicated. For example, a first target domain can be directly adjacent (i.e., contiguous) to a second target domain, or the first and second target domains may be separated by an intervening target domain. Assuming a 5′ to 3′ orientation of a target sequence, a first target domain may be located either 5′ to a second target domain, or 3′ to a second domain.

If desired, a target sequence may further comprise an additional moiety such as one partner of a ligand-binding pair, in order to facilitate binding to a detection probe comprising a nanoparticle attached to the other partner of the ligand-binding pair. For example, the target sequence may comprise a biotin moiety, which will facilitate binding to a detection probe comprising a nanoparticle attached to streptavidin. The biotin moiety may be incorporated into the target sequence using amplification methods that are analogous to known methods used to incorporate fluorescent moieties into target molecules, as set forth in more detail below.

Nucleic acid sequences of any practical length can be used as a target sequence. Generally, a target sequence is between ten and 50 nucleotides in length, and thus target sequences of ten, 15, 20, 25, 30, 35, 40, 45 or more nucleotides can be employed. However, target sequences of any length can be employed in the methods of the present invention, and in some cases may be shorter than ten nucleotides and longer than 50 nucleotides. For example, target sequences may be 60 nucleotides long, 75 nucleotides long, 85 nucleotides long, 100 nucleotides long, 300 nucleotides long, or even longer. If desired by the artisan, a target sequence may be fragmented prior to hybridization steps by using enzymatic, mechanical or other means as known in the art.

In some embodiments, target sequences can be isolated from biological samples, including, but not limited to, bodily fluids (e.g., blood, urine, serum, lymph, saliva, anal and vaginal secretions, perspiration semen, etc., of virtually any organism); environmental samples (e.g., air, plant, agricultural, water and soil samples); and research samples (i.e. amplification reaction products, purified samples such as purified genomic nucleic acids, and unpurified samples of bacteria, virus, genomic DNA, etc.).

If required, the target nucleic acid can be isolated from source biological samples using known techniques. For example, samples can be collected and concentrated or lysed, as required. Appropriate adjustment of pH, treatment time, lytic conditions and sample modifying reagents can also be made in order to optimize reaction conditions. Such modification techniques are well known to those of skill in the art.

Methods for nucleic acid isolation and purification can comprise simultaneous isolation of, for example, total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

Methods for nucleic acid isolation can optionally be optimized to promote recovery of pathogen-specific nucleic acids. In some organisms, for example fungi, protozoa, gram-positive bacteria, and acid-fast bacteria, cell lysis and nucleic acid release can be difficult to achieve using general procedures, and therefore a method can be chosen that creates minimal loss of the pathogen subset of the sample.

Semi-automated and automated extraction methods can also be used for nucleic acid isolation, including for example, the SPLIT SECOND™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith (1998) The Scientist 12(14):21-24 and Paladichuk (1999) The Scientist 13(16):20-23.

In some embodiments, a target nucleic acid comprises a double-stranded nucleic acid. Double stranded nucleic acid sequences can be prepared, for example, by isolating a double stranded segment of DNA. Alternatively, multiple copies of single stranded complementary oligonucleotides can be synthesized and annealed to one other under appropriate conditions. In order to provide a single-stranded target for hybridization, double-stranded nucleic acids are preferably denatured before hybridization. The term “denaturing” refers to the process by which strands of oligonucleotide duplexes are no longer base-paired by hydrogen bonding and are separated into single-stranded molecules. Methods of denaturation are well known to those skilled in the art, and include thermal and alkaline denaturation.

RNA isolation methods are known to one of skill in the art. See, Albert et al. (1992) J Virol 66:5627-2630; Busch et al. (1992) Transfusion 32:420-425; Hamel et al. (1995) J Clin Microbiol 33:287-291; Herrewegh et al. (1995) J Clin Microbiol 33:684-689; Izraeli et al. (1991) Nuc Acids Res 19:6051; McCaustland et al. (1991) J Virol Methods 35:331-342; Nataraian et al. (1994) PCR Methods Appl 3:346-350; Rupp et al. (1988) BioTechniques 6:56-60; Tanaka et al. (1994) J Gen Virol 75:2691-2698; and Vankerckhoven et al. (1994) J Clin Microbiol 30:750-753.

When mRNA is selected as a target sequence, the methods described herein can enable an assessment of pathogen gene expression. For example, detecting a pathogen in a biological sample can comprise determination of expressed virulence factors, other deleterious agents produced by a pathogen, or biosynthetic enzymes that generate virulence or other harmful pathogen gene products. Such analysis can facilitate distinction between active and latent infection, and indicate severity of an infection.

One of the advantages of the sandwich assay embodiments described herein is that the need to use nucleic acid amplification technology, cell culture, or other methods of selectively amplifying a target nucleic acid sequence is greatly diminished or even eliminated. However, while amplification steps are generally not required, procedures that include amplification prior to carrying out the detection methods of the present invention can be desirable in some cases. Nucleic acid “amplification” generally includes methods such as polymerase chain reaction (PCR), ligation amplification (or ligase chain reaction, LCR) and amplification methods based on the use of the enzyme Q-beta replicase. These methods are well known and widely practiced in the art., and reagents and apparatus for conducting them are commercially available.

Other amplification techniques are known in the art and may be used in conjunction with the detection methods described herein. These methods include random-primed PCR(RP-PCR); linker/adaptor-based DNA amplification; sequence-independent, single-primer amplification (SISPA); whole genome PCR; primer-extension pre-amplification (PEP); transcription-based amplification (variously called self-sustaining sequence replication, nucleic acid sequence-based amplification (NASBA), or transcription-mediated amplification (TMA)), amplified antisense RNA (aRNA); global RNA amplification, and others. See, e.g., Kinzler & Vogelstein (1989) Nuc Acids Res 17(10):3645-3653; Peng et al. (1994) J Clin Pathol 47:605-608); Reves & Kim (1991) Mol Cell Probes 5:473-481; Van Gelder et al. (1990) Proc Natl Acad Sci USA 87:1663-1667; Wang et al. (2000) Nat Biotech 18(4):457-459; Podzorski et al. in Murray et al., eds., Manual of Clinical Microbiology (American Society for Microbiology, Washington, D.C. (1995) p. 130); Zhang et al. (1992) Proc Natl Acad Sci USA 89:5847-5851; and U.S. Pat. No. 6,066,457 to Hampson et al.

In accordance with the methods described herein, any one of the above-mentioned amplification methods or related techniques can be employed to amplify the target nucleic acid sample and/or target sequence, if desired. In addition, such methods can be optimized for amplification of a particular subset of nucleic acid (e.g., genomic DNA versus RNA), and representative optimization criteria and related guidance can be found in the art. See, e.g., Cha & Thilly (1993) PCR Methods Appl 3:S18-S29; Linz et al. (1990) J Clin Chem Clin Biochem 28:5-13; Robertson & Walsh-Weller (1998) Methods Mol Biol 98:121-154; Roux (1995) PCR Methods Appl 4:S185-S194; Williams (1989) BioTechniques 7:762-769; and McPherson et al., PCR 2: A Practical Approach (IRL Press, New York, N.Y. (1995)).

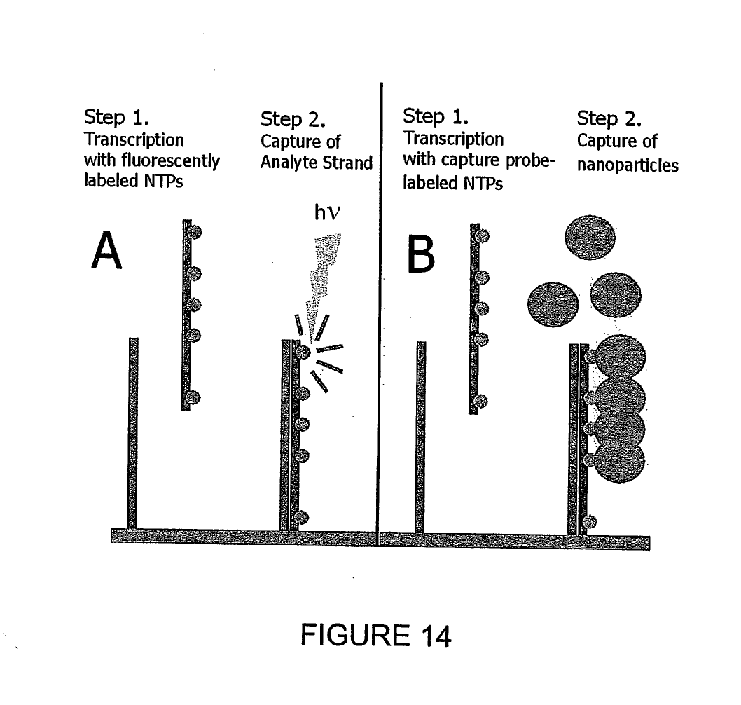

In some embodiments, amplification techniques are used to incorporate labeling or tagging moieties into a target sequence, which moieties are used to facilitate binding to a detection probe. In some embodiments, a target nucleic acid comprises a nucleic acid labeled or tagged with one partner of the ligand-binding pair (e.g., biotin), while a detection probe comprises a nanoparticle attached to the other partner of the ligand-binding pair (e.g., streptavidin). FIGS. 14A and 14B illustrate one method by which a ligand-binding pair moiety such as biotin can be incorporated into a target sequence. FIG. 14A schematically illustrates a known method of incorporating a fluorescent label into a target nucleic acid, in which a target is amplified using fluorescently-labeled nucleotide triphosphates (NTPs). In some embodiments of such a method, a target sequence is, for example, mRNA, and the complement of the target is enzymatically synthesized by means of a reverse transcriptase to produce a fluorescently-labeled cDNA target strand. Upon binding (hybridization) of a detection probe, the hybridization complex is exposed to light and detected by fluorescent detection and imaging means.

FIG. 14B illustrates a method useful in the practice of the present methods, by which biotin-tagged (rather than fluorescently-labeled) NTPs are incorporated into a cDNA target strand, and then used to hybridize nanoparticles coated with streptavidin. Methods of incorporating label and tag moieties (e.g., fluorescent labels, biotin, etc.) into target sequences using transcriptase-based amplification methods are known in the art. See, e.g., U.S. Pat. Nos. 6,589,737; 6,046,038; 6,004,755; 6,203,989; 6,589,742 and 6,503,711.

Thus, in some embodiments, a target sequence incorporates biotin moieties during an amplification reaction in which single stranded (ss) nucleic acid (e.g., mRNA) is used as a template, and nucleotides labeled with biotin are enzymatically incorporated into a complementary cDNA strand using a transcriptase (e.g., reverse transcriptase).

B. Probes

The term “probe,” as used herein, indicates a structure, complex or molecule having a capacity to selectively or substantially hybridize to a complementary target sequence in a heterogeneous mixture of nucleic acid molecules. In some embodiments, probes comprise oligonucleotide molecules. Oligonucleotide probes are typically designed to hybridize to target sequences in order to determine the presence or absence of the target sequence in a sample. As such, oligonucleotide probes as used in the methods described herein are generally designed to be complementary, in whole or in part, to a target sequence, such that hybridization between the target sequence and the probe or probes occurs.

The term “complementary sequences”, as used herein, indicates two nucleotide sequences that comprise antiparallel nucleotide sequences capable of pairing with one another upon formation of hydrogen bonds between base pairs. Additionally, the term “complementary sequences” means nucleotide sequences that are substantially complementary, as can be assessed by hybridization to the nucleic acid segment in question under relatively stringent conditions such as those described herein. The term “complementary sequence” also includes a pair of nucleotides that bind a same target nucleic acid and participate in the formation of a triplex structure as described, for example in U.S. Pat. No. 6,027,893 to Ørum et al. This complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence.

In some embodiments, each probe comprises at least one oligonucleotide sequence, which is complementary to a contiguous nucleic acid sequence of a target sequence such that the oligonucleotide sequence specifically hybridizes to the target sequence under stringent conditions, as defined herein.

The total length of a probe oligonucleotide will vary depending on its use, the length of the target sequence, and the hybridization and wash conditions. In general, oligonucleotide sequences of 5 to 50 nucleotides can be used; however, shorter or longer sequences can, in certain instances, be employed. In some cases, longer probes may be used, e.g. from about 50 to about 200-300 nucleotides or even longer in length.

In some embodiments, single-stranded DNA is used as an oligonucleotide component of the probes used in the present methods. In some embodiments, two oligonucleotides complementary to separate, non-overlapping segments, regions or domain of a target nucleic acid sequence are used in the sandwich hybridization format. In this embodiment, one of the oligonucleotides is used as a capture probe, while the other comprises the oligonucleotide component of the corresponding detection probe. By using two non-overlapping, non-complementary probes to identify a target nucleic acid sequence, the risk of “background noise” being interpreted as a false positive reading is reduced as compared to a system that relies on the hybridization of a single probe for detection.

Methods of making oligonucleotides of a predetermined sequence are well-known. See, e.g., Sambrook et al., supra, and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides. Oligoribonucleotides and oligodeoxyribonucleotides can also be prepared enzymatically.

### III. Electrode Materials

As used herein, the term “electrode” means a composition which is able to carry or sense an electrical current or charge, and then convert it to a measurable signal. In some embodiments, an electrode is a solid substrate comprising a conducting material or a semiconducting material.

Electrode material can be selected according to desired redox potential range, ease of surface attachment of nucleic acid to surface, and appropriate or desired optical properties. As provided above, one limitation in the selection of an electrode material is that it cannot be identical to the material that the detection probe nanoparticle comprises. Electrode materials include, but are not limited to, certain metals and their oxides, such as gold, platinum, palladium, aluminum, indium tin oxide (ITO), tin oxide, fluorine-doped tin oxide, cadmium oxide, iridium oxide, ruthenium oxide, zinc tin oxide, antimony tin oxide, platinum oxide, titanium oxide, palladium oxide, aluminum oxide, molybdenum oxide, tungsten oxide, and others. In one particular embodiment, the electrode comprises indium tin oxide (ITO).

The electrode can comprise a single conductive material or multiple conductive materials. The conductive electrode material can be layered over a second material, such as a polymer or otherwise non-conducting surface. In some embodiments, the electrode is formed on a solid, non-conducting substrate. The substrate can comprise a wide variety of materials, including but not limited to glass, fiberglass, teflon, ceramics, silicon, mica, plastic (including acrylics, polystyrene and copolymers of styrene and other materials), polypropylene, polyethylene, polybutylene, polycarbonate, polyurethanes, TEFLON™, combinations thereof, and the like. Alternatively, a support can be constructed from a polymer material such as high density polyethylene (HDPE), often used in 96-well titer plates. In yet another example, a polyacrylamide gel can be employed as a solid support for the electrode (Dubiley et al., (1997) Nucleic Acids Res. 25: 2259-2265).

Solid substrates on which electrodes may be formed also include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g., all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

The electrodes described herein are depicted in the Figures as a flat surface. However, a flat surface is only one of the possible conformations of the electrode, and as such is illustrated for schematic purposes only. The conformation of the electrode will vary with the detection method used. For example, flat planar electrodes may be preferred for methods requiring addressable locations for detection.

In some embodiments, and as discussed in more detail herein, the electrode can optionally and further comprise a passivation agent. As used herein, the term “passivation” generally means the alteration of a reactive surface to a less reactive state. Passivation can refer to, for example, decreasing the chemical reactivity of a surface or to decreasing the affinity of a surface for nucleic acids. Stated differently, passivation is a method by which a surface is coated with a moiety having the ability to block subsequent binding to the surface at points where the moiety is bound.

In some embodiments, a passivation agent is in the form of a monolayer on the electrode surface. The efficiency of hybridization may increase when the detection probe is at a distance from the electrode. A passivation agent layer facilitates the maintenance of the probe away from the electrode surface. In addition, a passivation agent can serve to keep charge carriers away from the surface of the electrode. Thus, this layer can help to prevent direct physical or electrical contact between the electrodes and the nanoparticles of the detection probes, or between the electrode and charged species within the redox compound solution. Such contact can result in a direct “short circuit” or an indirect short circuit via charged species which may be present in the sample. Accordingly, the monolayer of passivation agents is preferably tightly packed in a uniform layer on the electrode surface, such that a minimum of “holes” exist.

IV. Modification of Electrode Surfaces with Capture Probes

In some embodiments, the electrode comprises a plurality of capture probes attached to the electrode surface in an array format. As used herein, the terms “nucleic acid microarray,” and “nucleic acid hybridization array” are used interchangeably, and mean an arrangement of a plurality of nucleic acid sequences (e.g., capture probes) bound to a support (e.g., an electrode). The terms “addressable array” and “array” are used interchangeably, and mean a plurality of entities arranged on a support in a manner such that each entity occupies a unique and identifiable position. In the methods described herein, the entities are capture probes (e.g., capture oligonucleotides) immobilized to the surface of an electrode. As used herein, the terms “immobilize” and “attach” are used interchangeably to mean a chemical and/or mechanical association of one moiety with one or more surfaces (e.g., solid surfaces). The association can be covalent or non-covalent, and can be direct or indirect.

In some embodiments, capture probes attached to the surface of an electrode are ordered such that each capture probe sample has a unique, identifiable location on the support. The physical location on the electrode where a capture probe is attached or immobilized is referred to herein as an “attachment point.” The identity of a capture probe bound to an electrode at a given location can be determined in several ways. One exemplary way to correlate a capture probe with its location is to attach the capture probe to the support at a known position (see, e.g., Pirrung, (1997) Chem. Rev. 97: 473-486). Discrete locations on the support can be identified using a grid coordinate-like system. In this approach, the working area of the support surface can be divided into discrete areas that may be referred to interchangeably as “spots” or “patches”. Different capture probes can subsequently be attached to the surface in an orderly fashion, for example, one capture probe, or one sample of identical capture probes, to a spot. In this strategy, the probe oligomers can be applied one or several at a time. In one exemplary method, sites at which it might be desirable to temporarily block probe binding can be blocked with a blocking agent. The blocking agent can be subsequently removed and the site freed for probe binding. This process can be repeated any number of times, thus facilitating the attachment of a known probe at a known location on a support.

Localizing capture probes to an electrode surface at known locations can involve the use of microspotting. In this approach, the location of the capture probes on an electrode surface is determined by the ordered application of probe samples in a group. That is, capture probes are ordered in known locations prior to application to the electrode surface. In this way, the location of each probe is known as it is applied. Appropriate devices for carrying out this approach are commercially available and can be used with the detection methods described herein. For example, the present methods are compatible with the commercially available GENECHIP™ system (Affymetrix, Inc., Santa Clara, Calif.) or the commercially available SPOTBOT™ Automated Spotting Arrayer (TeleChem International, Sunnyvale, Calif.).

As set forth above, in some embodiments a single-stranded nucleic acid sequence is used as a capture probe. For example, a capture probe can comprise a single-stranded cDNA sequence complementary to a target gene of interest or to a target domain thereof. The capture probe can be attached to the electrode surface indirectly via an “attachment linker,” as defined herein. In this embodiment, one end of an attachment linker is attached to a capture probe, while the other end (although, as will be appreciated by those in the art, it need not be the exact terminus for either) is attached to the electrode.

The method of attachment of the capture probe to the attachment linker can generally be done as known in the art, and will depend on the composition of the attachment linker and the capture probe. In general, the capture probe is attached to the attachment linker through the use of functional groups on each moiety that can then be used for attachment. Preferred functional groups for attachment are amino groups, carboxy groups, oxo groups and thiol groups. Using these functional groups, the capture probes can be attached using functional groups on the electrode surface.

In one example of an attachment approach suitable for attachment of capture probes to an electrode surface, one or more probe capture sequences are initially incubated with a solution of a thio-alcohol for a pre-selected period of time. In some embodiments, C6 mercaptohexanol is employed as a thio-alcohol, in accordance with techniques described by Loweth et al., (1999) Angew. Chem. Int. Edit. 38: 1808-12, and Storhoff & Mirkin, (1999) Chem. Rev. 99: 1849-62. Thio-alcohol and capture probe are added in amounts so as to bring the final concentration of capture probe in the solution to about 20% or less. The incubation time permits the covalent association of the 3′ end of the capture probe oligonucleotide with the hydroxyl group of the thio-alcohol. The solution is then exposed to the surface of a support under conditions that permit association of the sulfur atom of the thio group with the surface of the support. Suitable equipment is commercially available and can be used to assist in the binding of a target sequence to a support surface.

In another specific example, a monolayer of 12-phosphonododecanoic acid is formed on the electrode surface. The carboxylic acid of 12-phosphonododecanoic acid is then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K Kerman et al., Analytica Chimica Acta (2002) 462, 39-47; E. Huanq et al., Langmuir (2000) 16, 3272-3280; and G. T. Hermanson, Bioconjugate Techniques (1996) (Academic Press: San Diego). This activated carboxylic acid group is attacked by the primary amine (acting as a nucleophile) of a 5′-modified C₃H₂ single-stranded DNA strand to form an amide bond between the monolayer of 12-phosphonododecanoic acid and the 5′ modified C₃H₂ ssDNA.

Other functional groups useful for attaching oligonucleotides to solid surfaces (i.e., electrodes and nanoparticles) include, for example, moieties comprising thiols, carboxylates, hydroxyls, amines, hydrazines, esters, amides, halides, vinyl groups, vinyl carboxylates, phosphates, silicon-containing organic compounds, and their derivatives. Other functional groups useful for attachment include phosphorothioate groups (see, e.g., U.S. Pat. No. 5,472,881 for the binding of oligonucleotide-phosphorothioates to gold surfaces), aminosilanes (see, e.g., K. C. Grabar et al., J. Am. Chem. Soc. (1996) 118, 1148), and substituted alkylsiloxanes (see, e.g. Burwell, Chemical Technology 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides terminated with a 5′ thionucleoside or a 3′ thionucleoside can also be used for attaching oligonucleotides to solid electrode surfaces. The length of these attaching functional groups is chosen such that the conductivity of these molecules does not hinder electron transfer from the nanoparticle to the electrode via the hybridized probe and target nucleic acids. Stated differently, these functional groups are preferred to have higher conductivities than double-stranded nucleic acid.

In some embodiments, a “tag” or “linker” nucleic acid sequence can be employed to attach capture probes to electrode surfaces. When a tag sequence is employed, an electrode can comprise a tag nucleic acid complement. A tag complement is a sequence that is complementary to a tag sequence associated with a capture probe. Thus, when a capture probe comprising a tag sequence is contacted with an electrode comprising a tag complement under suitable hybridization conditions, a duplex can form.

A tag sequence can comprise, for example, a sequence that is complementary to a support-bound tag complement. A tag sequence can be associated with a target sequence, which can then be amplified by PCR prior to association with a nanoparticle. The PCR amplicon will comprise a nucleic acid sequence comprising the tag sequence and a target target sequence. The PCR amplicon then comprises a sequence that is complementary to a support-bound tag complement. Inclusion of a tag sequence, for example as a component of a target sequence, offers the advantage that a support need not be specific for a given target sequence, but rather can be universal in the sense that it is specific for a tag complement, but not for any particular target sequence. Thus, by employing a tag complement, an electrode (or nanoparticle, as described herein) can be independent of the source of a capture probe oligonucleotide (as to species, etc.) in the sense that the electrode can be specific for a tag sequence, but not for any particular capture probe sequence. Thus, by employing a method comprising the use of a tag-tag complement approach, the need to form different electrode supports for different probe and/or target sequences is mitigated. See, e.g., WO 94/21820, WO 97/31256, WO 96/41011 and U.S. Pat. No. 5,503,980.

Following attachment of a capture probe to the surface of the electrode, the areas of the electrode surface to which no probe is bound can be passivated, as defined above. A passivation process can be implemented after probes are bound to the support, and can include sequential synthesis and co-deposition approaches, as is known in the art.

In some embodiments, passivation is accomplished by exposing the surface to thio-alcohol, as described above. For example, the same thio-alcohol can be used to passivate the surface as was used in attaching the probe to the surface. In some embodiments, thio-alcohols of shorter or longer length than those used to attach capture probes can be employed.

In some embodiments, other molecules, i.e. “passivation moieties” can be used passivate the surface of a support. For example, polyethylene glycol (PEG), various alcohols and carboxylates can all be used to passivate the surface of a support, as can COO— and CONH₂ moieties. In some embodiments, passivation moieties can also be non-covalently or covalently attached. Indeed, virtually any material can be used to passivate a support surface, with the understanding that the passivation material must associate with the support to form a protective layer coating the support, and that the passivating process, which can be performed after a probe is already associated with the surface of the support, does not damage any probes already bound to the support. As described above, a passivation step can also be performed to reduce the potential for nonspecific association between a nanoparticle complex and a support.

### V. Other Components

Detection probes used in the practice of some embodiments of the presently described methods generally comprise at least two components. In some embodiments, the two components include an oligonucleotide nucleic acid sequence, and a nanoparticle to which the oligonucleotide is attached.

In some embodiments, a non-oligonucleotide ligand is used instead of an oligonucleotide sequence. In some embodiments, the non-oligonucleotide ligand is a member of a ligand-binding pair, and its other, corresponding member of the binding pair is attached to or incorporated into the target sequence, such that the target sequence can specifically or selectively bind the detection probe. In one example of these embodiments, a target sequence is biotinylated according to methods described above (e.g., nucleic acid amplification incorporating biotin-tagged nucleotides), while a detection probe comprises a nanoparticle coated with streptavidin. Methods for attaching streptavidin to nanoparticles are known, see, e.g., Shaiu et al., Nuc. Acids Res. 21, 99 (1993).

Detection probes may also and optionally comprise other useful moieties, including electrochemically-active redox reaction mediators, catalysts, supplementary labeling molecules (e.g., fluorescent, magnetic or chemiluminescent moieties), detection enhancers, and the like.

As used herein, the terms “nano”, “nanoscopic”, “nanometer-sized”, “nanostructured”, “nanoscale”, and grammatical derivatives thereof are used synonymously, and in some cases interchangeably. As used herein, the term “nanoparticle” can refer to a component to which a nucleic acid is bound. Typically, but not necessarily, a nanoparticle is an approximately spherical metal atom-comprising entity. In one example, a nanoparticle is a particle comprising a material such as a metal, a metal oxide or a semiconductor. In other examples, a nanoparticle can comprise a polymeric species or any other conducting material.

Nanoparticles are generally less than about 1000 nanometers (nm) in diameter, usually less than about 200 nanometers in diameter and more usually less than about 100 nanometers in diameter. In certain particular embodiments, nanoparticles are between about 10 nm and 20 nm in diameter, while in other embodiments, the size of the nanoparticle is less than about 10 nm. Representative ranges of nanoparticle sizes include but are not limited to from about 5 to about 200 nanometers, from about 5 to about 100 nanometers, from about 5 to about 50 nanometers, from about 5 to 20 nanometers, from about 10 to about 200 nanometers, from about 10 to about 100 nanometers, and from about 10 to about 50 nanometers.

A nanoparticle can comprise almost any material, as long as the material is different from the electrode material used in the hybridization reactions. In the practice of the methods described herein, materials that can be used in nanoparticle fabrications are able to catalyze electrochemical reactions, and/or are able to function as a bridge for electrons between redox mediators in solution and an electrode, and/or are able to alter the rate of electron transfer at an electrode. As such, one consideration when selecting a material for a nanoparticle is the chemical reactivity profile of the material. The chemical reactivity profile of a material is a consideration because other entities, such as oligonucleotides or ligand binding pair components such as strepatavidin, will ultimately be associated with the nanoparticle.

Additionally, it can be desirable to associate an additional, secondary component with a nanoparticle. Exemplary secondary components include, but are not limited to, electrochemically-active moieties (e.g., ruthenium complexes), catalysts, supplementary labeling molecules (e.g., fluorescent, magnetic or chemiluminescent moieties), and detection enhancers. Therefore, the reactivity of a nanoparticle to a desired secondary component can also be a consideration. Thus, considerations when selecting and/or designing a nanoparticle can include size, material, chemical reactivity of the material the ease with which an oligonucleotides can associate with the nanoparticle, and the ease with which a secondary component can associate with the nanoparticle.

Metals, metal oxides, conductive polymers, dendrimers (e.g. branched dendrimers) and semiconductors are examples of some materials that can be employed in the fabrication of a nanoparticle that can be used as a component of a detection probe. Nanoparticles can be formed from metals and metal oxides, including but not limited to gold, silver, titanium, titanium dioxide, tin, tin oxide, iron, iron^{III} oxide, copper, nickel, aluminum, steel, indium, platinum, indium tin oxide, fluoride-doped tin, ruthenium oxide, germanium cadmium selenide, cadmium sulfide and titanium alloy. Nanoparticles can also be formed from semiconductor materials (e.g., CdSe, CdS, and CdS or CdSe coated with ZnS) and magnetic (e.g., ferromagnetite) colloidal materials. Nanoparticles may comprise ZnS, ZnO, TiO₂, AgI, AgBr, HgI₂, PbS, PbSe, ZnTe, CdTe, In₂S₃, In₂ Se₃, Cd₃P₂, Cd₃ As₂, InAs, and GaAs, as is known in the art.

In a particular example, the nanoparticle material comprises one or more of gold, silver and platinum or combination or alloy of any of the foregoing. As used herein, the term “gold” means element 79, which has the chemical symbol Au; “silver” means element 47, which has the chemical symbol Ag, and “platinum” means element 78, which has the chemical symbol Pt.

Nanoparticles comprising the above-listed materials are generally available commercially from numerous suppliers, including but not limited to Vacuum Metallurgical Co., Ltd. (Chiba, Japan), Vector Laboratories, Inc. (Burlingame, Calif.), Ted Pella, Inc., Amersham Corporation and Nanoprobes, Inc.

Nanoparticles can also be fabricated if desired, using any method. See, e.g., Marinakos et al. (1999) Adv. Mater. 11:34; Marinakos et al. (1998) Chem. Mater. 10:1214-19; Enustun & Turkevich (1963) J. Am. Chem. Soc. 85:3317; Havashi (1987) J. Vac. Sci. Technol. A5(4): 1375-84; Hayashi (1987) Phys. Today, December 1987, 44-60; MRS Bulletin, January 1990, pp. 16-47; G. Schmid, (ed.) Clusters and Colloids (V C H, Weinheim, 1994); M. A. Havat (ed.) Colloidal Gold: Principles, Methods, and Applications (Academic Press, San Diego, 1991); R. Massart, IEEE Transactions On Magnetics, 17, 1247 (1981); T. S. Ahmadi, et al., Science, 272, 1924 (1996); A. Henglein, et al., J. Phys. Chem., 99, 14129 (1995); A. C. Curtis, et al., Angew. Chem. Int. Ed. Engl., 27, 1530 (1988); Weller, Angew. Chem. Int. Ed. Engl., 32, 41 (1993); Henglein, Top. Curr. Chem., 143, 113 (1988); Henglein, Chem. Rev., 89, 1861 (1989); Brus. Appl. Phys. A., 53, 465 (1991); Bahncmann, in Photochemical Conversion and Storage of Solar Energy (eds. Pelizetti and Schiavello 1991), page 251, and others.

Special metal coated particles known as “nanoshells” are also included in the definition of the term “nanoparticles,” in the practice of the present methods. In general, nanoshells comprise a non-conducting, semiconductor or dielectric core coated with an ultrathin metallic layer. In general, nanoshells have diameters ranging from a few nanometers up to about 5 microns, and have defined wavelength absorbance maxima across the visible and infrared range of the electromagnetic spectrum. Gold nanoshells are one class of optically active nanoparticles that consist of a thin layer of gold surrounding a dielectric core, such as gold sulfide (see, e.g., R. D. Averitt et al., J. Opt. Soc. Am. B 16:1824-1832 (1999), and R. D. Averitt et al., Phys. Rev. Lett. 78:4217-4220 (1997)), or other materials.

### VI. Detection Probes: Attachment of Binding Partners to Nanoparticles

In general, the method described above in reference to attaching oligonucleotides to electrode surfaces can also be used to attach oligonucleotides to nanoparticle components of detection probes. For instance, oligonucleotides functionalized with alkanethiols at their 3′-termini or 5′-termini readily attach to gold nanoparticles. See, e.g., Whitesides, Proceedings of the Robert A. Welch Foundation 39th Conference On Chemical Research Nanophase Chemistry, Houston, Tex., pp. 109-121 (1995); Mucic et al., Chem. Commun. (1996) 555-557.

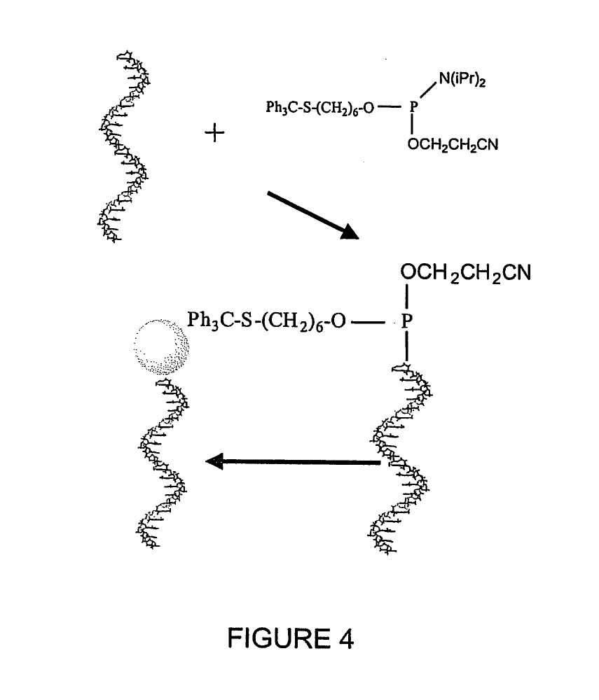

When attaching an oligonucleotide probe to a nanoparticle, a thiolation reaction can be performed to add a thiol group to the 5′ end of a single-stranded oligonucleotide. Alternatively, an amination reaction can be performed and will proceed mutatis mutandis with the thiolation reaction described herein. The general purpose of the reaction is to introduce a nucleophilic center that can subsequently be functionalized with a nanoparticle as described herein. As shown in FIG. 4 and immediately below, a suitable thiol modifier phosphoramidite reagent is the following compound, which is available from Glen Research, Corp. of Sterling, Va.:

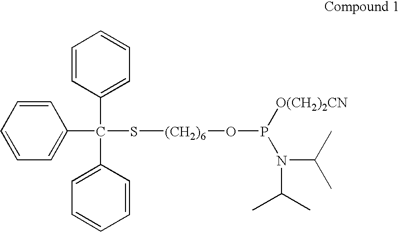

Referring now to FIG. 4, single-stranded oligonucleotides are incubated with a thiol modifier phosphoramidite under anhydrous conditions that permit attachment of the phosphine to the 5′ end of the oligonucleotide. The reaction can be carried out in a nucleic acid synthesizer under standard (and anhydrous) conditions. The thiol modifier is generally added in the last step of synthesis of an oligonucleotide. The phosphine is oxidized using iodine, and the purification is generally the same as that used for unlabeled oligonucleotides. In this reaction, the thiol group is generally protected by a protecting trityl or acetic thioester group and is separated from the 5′-phosphodiester by a variable-length carbon linker. A six-carbon linker is represented in the structure of Compound 1.

The oligonucleotide complex is then subjected to thiol deprotection to remove the trityl group. Specifically, the protecting trityl group is removed by treatment with silver nitrate and dithiothreitol (DTT). The oligonucleotide complex is then incubated with a nanoparticle. The two entities are joined at the thiol exposed by the removal of the trityl group during the deprotection reaction. The formed nanoparticle-oligonucleotide conjugates (i.e., detection probes) can be maintained in the reaction vessel until use.

When a non-synthetic (i.e. isolated, extended or reverse transcribed) oligonucleotide is employed as a component of the detection probe in the present invention, the oligonucleotide can be attached to a nanoparticle in a variety of ways. One mechanism for attaching a non-synthetic oligonucleotide probe to a nanoparticle, generally described as an “end-labeling” scheme, involves derivatizing the 5′ hydroxyl group of an oligonucleotide to incorporate a functional group reactive with the nanoparticle material on the 5′ end of the oligonucleotide. A representative, but non-limiting, list of functional groups includes carboxylate groups, amine groups and thiols group. Such functional groups can be added to an oligonucleotide as a step in the synthesis of the oligo and can be programmed as an additional step in automated nucleic acid synthesizers, as is known in the art.

In some embodiments of an attachment scheme, an oligonucleotide having a 5′ hydroxyl group is incubated under suitable anhydrous reaction conditions with N,N′ carbonyldiimidazole and subsequently with a cysteamine, thereby end labeling the oligo with a thiol group according to Reaction Scheme 1:

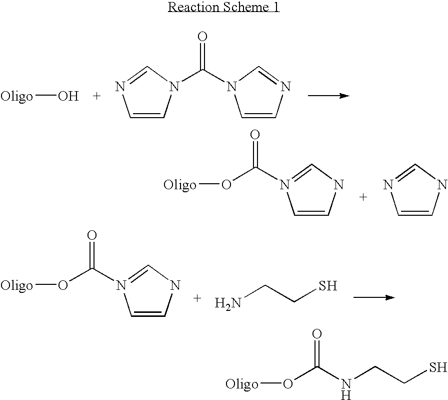

In some embodiments of an attachment scheme, a carboxylate (or a thiol, amine or any other moiety) moiety can be chemically incorporated into a reverse transcription reaction or, as noted, attached to the 5′ hydroxyl of a synthesized oligonucleotide. Similarly, phosphonates and amines can be employed to attach an oligonucleotide to a metal oxide central component or a nanoparticle. Cystamine-based attachment strategies can also be employed. Those of ordinary skill in the art can recognize reaction conditions that might be damaging to an oligonucleotide and can design attachment strategies, using the above disclosure as a guide, so as to maintain the integrity of the oligonucleotide. It is noted that a deoxynucleotide phosphate (dNTP) having a 5′ hydroxyl group can also be derivatized using Reaction Scheme 1 for attachment to a nanoparticle. Suitable protective groups and additional reaction conditions can be employed, and are known to those of skill in the art.

Although the examples provided above illustrate the attachment of one moiety (i.e., an oligonucleotide) to one nanoparticle, the present methods specifically encompass embodiments in which a plurality of moieties are attached to a single nanoparticle (i.e., the nanoparticles of the present methods are polyvalent). In some embodiments, a plurality of identical oligonucleotides is attached to one nanoparticle. In some embodiments, one or more identical oligonucleotide sequences are attached to the nanoparticle, as well as one or more other, non-oligonucleotide embodiments (e.g., one or more electrochemically active moieties, labels, tags, ligand-binding pair components, etc., as previously described herein). Alternatively, and as explained above, nanoparticles can be attached to non-oligonucleotide components entirely, as in the case of a detection probe comprising a nanoparticle attached to ligand-binding pair components such as streptavidin.

### VII. Sandwich Format Hybridization Assays

After a capture probe has been immobilized to an electrode surface, a target nucleic acid has been selected and a detection probe comprising a nanoparticle has been prepared, a series of hybridization reactions are performed. In some embodiments, the hybridization reactions are carried out in a sandwich assay format. Generally, a target sequence is brought into contact with an electrode whose surface has been modified by attaching capture probes to the electrode surface. The target sequence may be brought into contact with the capture probe under hybridization conditions in any suitable manner. In some embodiments, the target sequence is in solution, and the electrode having the capture probe immobilized thereon is immersed into the solution containing the target sample. In some embodiments, the solution is a biological sample.

If the capture oligonucleotide and the target nucleic acid comprise complementary sequences, the target sequence will hybridize with the capture probe, thus forming a first hybridization complex comprising a capture probe and a target sequence. After capture and target nucleic acids have been permitted to hybridize, any unbound (unhybridized) nucleic acid can be removed from the surface of the electrode after the hybridization reaction.

In some embodiments, the capture probes attached to the electrode have sequence complementary to a first domain of the target sequence to be detected. The target sequence is contacted with the capture probe under conditions effective to allow hybridization of the capture probe with the target. In this manner, the target becomes bound to the capture probe. Any unbound target sequence can optionally be removed from the electrode before adding detection probe, as defined herein.

To complete the sandwich assay, the electrode surface (with capture probe-target sequence hybridization complexes attached thereto) is brought into contact under hybridization conditions with a detection probe sample comprising nanoparticle-oligonucleotide conjugates. The detection probe may be present in a solution, which can be dispensed onto the electrode surface. Alternatively, the electrode surface can be immersed into the solution comprising the detection probe. If a first hybridization complex has formed at a location on the electrode, the oligonucleotide component of the detection probe will hybridize to the target sequence component of the first hybridization complex, thus forming a second hybridization complex comprising a capture probe, a target sequence and a detection probe comprising a nanoparticle, which second hybridization complex is attached to the electrode surface by means of the capture probe. The hybridization steps can be performed in any order, or simultaneously, with or without intervening wash steps.

In some embodiments, the oligonucleotide component of the detection probe has sequence complementary to a second domain of the target nucleic acid, and the contacting takes place under conditions effective to allow hybridization of the oligonucleotides attached to the nanoparticle to the target sequence. In this manner, the detection probe nucleic acid-nanoparticle conjugates become attached to the electrode as part of a hybridization complex. After the detection probe has been hybridized to the target, unbound nanoparticle-oligonucleotide conjugates can be removed from the electrode.

Thus, the methods described herein utilize capture and detection probes that substantially hybridize to a target sequence. The phrases “hybridizing substantially to” and “substantially hybridizes” refer to complementary hybridization between a probe nucleic acid molecule and a target nucleic acid molecule, and embraces hybridization of substantially identical sequences that can be accommodated by adjusting the stringency of the hybridization media to achieve the desired hybridization.

The terms “specifically hybridizes” and “selectively hybridizes” each refer to binding, duplexing, or hybridizing of a molecule only to a particular nucleotide sequence under stringent conditions when that sequence is present in a complex or heterogeneous nucleic acid mixture.

An extensive guide to the hybridization of nucleic acids is found in Tijssen, Laboratory Techniques in Biochemistry and Molecular Biology-Hybridization with Nucleic Acid Probes (Elsevier, New York, N.Y. (1993) Part I, Chapter 2). A variety of hybridization conditions may be used in the present invention, including high, moderate and low stringency conditions; see for example Maniatis et al., supra, and Ausubel, et al., supra. The hybridization conditions may also vary when a non-ionic backbone, i.e. PNA is used, as is known in the art.

Stringent conditions are those that allow hybridization between two nucleic acid sequences with a high degree of homology, but preclude hybridization of random, non-complementary sequences. In general, hybridization at low temperature and/or high ionic strength is termed low stringency, and hybridization at high temperature and/or low ionic strength is termed high stringency. The temperature and ionic strength of a desired stringency are understood to be applicable to particular lengths of nucleic acid sequences, to the base content of the sequences, and to the presence of other compounds such as formamide in the hybridization mixture.

Stated otherwise, “stringent hybridization conditions” and “stringent hybridization wash conditions,” in the context of nucleic acid hybridization experiments, are both sequence- and environment-dependent. In general, longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

One can employ varying conditions of hybridization to achieve varying degrees of selectivity of probe towards target sequence, with the general rule that the temperature remain within approximately 10° C. of the duplex's predicted Tₘ, which is the temperature (under defined ionic strength and pH) at which 50% of the target sequence hybridizes to a perfectly matched probe. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. A high stringency wash can optionally be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C.

For shorter sequences (e.g., about 10 to 50 nucleotides), stringent conditions typically involve incubation in salt concentrations of less than about 1.0 M sodium ion, typically about 0.01 to 1.0 M sodium ion (or other ion) concentration, at pH 7.0-8.3, at a temperature of at least about 30° C.

For applications requiring high selectivity, one will typically desire to employ relatively stringent conditions to form hybridization complexes, e.g., conditions of high stringency where one will select relatively low salt and/or high temperature conditions, such as provided by about 0.02 M to about 0.15 M NaCl at temperatures of about 50° C. to about 70° C. Such selective conditions tolerate little, if any, mismatch between the probe and the target strand.

It is generally appreciated that conditions can be rendered more stringent by the addition of increasing amounts of formamide, which serves to destabilize the hybridization complex in the same manner as increased temperature. Thus, hybridization conditions can be readily manipulated, and conditions can be readily selected depending on the desired results.

### VIII. Electrochemical Reactions

The electrode surface and any hybridization complexes bound thereto are subsequently or simultaneously exposed to a redox solution comprising a redox mediator. The redox solution, in some embodiments, further comprises an electrolyte (i.e., is an electrolyte solution comprising a redox mediator). In some embodiments, the assembly comprising the electrode and any attached hybridization complexes is immersed into a redox solution comprising a redox mediator, and an electrolyte. Alternatively, the hybridization reactions of target sequences to the capture and detection probes are carried out in the presence of the redox solution. In some embodiments, hybridization steps and exposure to the redox solution occur simultaneously and/or are carried out in the same reaction chamber.

As used herein, the terms “redox mediator” and “redox compound” are used interchangeably and mean a redox-active molecule or part of a molecule that is capable of undergoing changes in its electronic properties. The terms “redox-active moiety” or “redox-active molecule” refers to a compound that can be oxidized and reduced, i.e., which contains one or more chemical functions that accept and transfer electrons. Redox-active mediators and compounds are electroactive, where the term “electroactive” means that the compound has the ability to change electronic configuration. The term refers to a molecule or structure and includes the ability to transfer electrons, the ability to act as a conductor of electrons and the ability to act as an electron donor or acceptor.

Redox mediators are thus chemical species capable of being reduced and/or oxidized. Redox mediators include, but are not limited to, metals, metals ions and complexes thereof that are capable of being reduced and/or oxidized; organic compounds capable of being reduced and/or oxidized; and inorganic compounds capable of being reduced and/or oxidized.

In some embodiments, the redox mediator comprises a redox couple. Redox couples are analytes differing only in oxidation state. By way of example, a redox couple that can be used in the present methods is ferricyanide/ferrocyanide, or Fe(CN)₆³⁻/Fe(CN)₆⁴⁻. The anion, Fe(CN)₆³, contains an iron atom in the +3 oxidation state. At the surface of an electrode, a single electron can be added to the ferricyanide anion. This causes it to be reduced to the anion, Fe(CN)₆⁴⁻, which contains an iron atom in the +2 oxidation state. Other redox couples include ferriin-ferroin, ferrocene/ferrocinium, EDTA/EDTA⁻¹, H₂O/H₂, and O₂/H₂O₂.

In some embodiments, the redox mediator is an inorganic redox couple, generally employing an iron or ruthenium couple. The iron may be in any convenient form, and in a particular embodiment is coordinated, such as with hexacyanoferrate, ferricyanide/ferrocyanide, ferriin-ferroin, ferrocene/ferrocinium, or other stable form of iron that is capable of undergoing one-electron transfer.

In some embodiments, the redox mediator is a metallocene or a derivative thereof. In a particular embodiment, the redox mediator is a ferrocene (such as ferrocene itself), or a derivative thereof. A ferrocene has, as its fundamental structure, an iron atom held “sandwiched” by dative bonds between two pentadienyl rings. It is an electroactive organometallic compound, acting as a pH-independent reversible one-electron donor. The electrochemistry of ferrocene has been characterized. See, e.g., Uosaki et al., (1991) Langmuir 7: 1510; Chidsey et al., (1990) J. Am. Chem. Soc. 112: 4301; Tender et al., (1994) Anal. Chem. 66: 3173.

Suitable redox mediators thus include ferrocene and its derivatives, which include 1,1′-ferrocene dicarboxylic acid, 1,1′-dimethylferrocene (DMF), polyvinylferrocene (having monomeric ferrocene or a monomeric ferrocene derivatives such as (ferrocene)₄ and “boron tetraferrocene” or [B(ferrocene)₄)]), [N-ferrocenoyl]-4-aminophenyl phosphate, and ferrocene monocarboxylic acid (FMCA).

Alternative redox mediators can be selected from the groups including but not limited to quinones (e.g., benzoquinone), phenylene diamines, metal complexes with organic ligands tetracyanoquinodimethane, N,N,N′,N′-tetramethyl-p-phenylenediamine, 2,6-dichloroindophenyl phosphate, tetrathiafulvalene, coordinated ruthenium compounds, carboranes, conductive salts of tetracyanoquinodimethane (TCNQ), haloanils and derivatives thereof, vologens, alkyl substituted phenazine derivatives, 3,3′,5,5′-tetramethylbenzidine, bis-cyclo pentadienyl complexes of transition metals; and phenol derivatives including ferrocene-phenol and indophenol compounds.

Preferred redox mediators facilitate slow redox at the electrode surface, and fast redox at the nanoparticle. Selection of the appropriate electrolyte solution can be made according to known parameters. In a particular embodiment, the redox mediator is the EDTA/EDTA⁻¹ redox couple. In some embodiments, the redox mediator is the H₂O/H₂ redox couple. In some embodiments, the redox mediator is the O₂/H₂O₂ redox couple.

The redox mediator can be present in solution at any appropriate concentration, for example in the range of about 1.0 to about 1000 μM, and from about 10 to about 200 μM, optionally depending on the selection of the mediator.

Electrochemical contact is advantageously provided using a conducting electrolyte solution in contact with each of the electrodes or microelectrode arrays of the invention. An electrolyte solution can made by adding an ionic salt to an appropriate solvent.

Electrolyte solutions useful in the apparatus and methods of the invention include any electrolyte solution at physiologically-relevant ionic strength (equivalent to about 0.15M NaCl) and neutral pH (e.g., pH 7.0 to 7.6). The salt must become fully dissociated in the solvent in order to generate a conducting (i.e., ionic) solution.

Electrolyte solutions can be aqueous or non-aqueous. A wide range of salts can be used for aqueous electrolyte solutions. Suitable solvents include water, various alcohols, acetonitrile, DMF, DMSO, THF, methylene chloride, propylene carbonate, and others. Since the redox potentials of some compounds are pH sensitive, buffered solutions should be used for these compounds. Various buffers may be employed in the electrolyte solution, which include but are not limited to tris-(hydroxymethyl) methylamine (Tris), phosphate, borate, or the like, usually employing a buffer suitable for the particular enzyme system. Non-limiting examples of electrolyte solutions useful with the methods described herein include but are not limited to saline, phosphate buffered saline, potassium nitrate, HEPES buffered solutions, and sodium bicarbonate buffered solutions.

### IX. Detection of Electrochemical Reaction

As used herein, the term “detect” means determining the presence and/or amount of a target molecule, entity or event. Determination is carried out by observing the occurrence of a detectable signal (e.g., an electrical, chemical, visual or spectroscopic signal) that occurs in the presence of the target molecule or entity, or during the occurrence of the target event (i.e., a hybridization event). Determination can be qualitative (i.e., detecting mere presence or absence, or detecting relative amounts), or can be quantitative (i.e., specific amounts are measured or quantitated).

As used herein, the term “electrical current” means the movement of electrons from a higher energy level to a lower energy level. Generally, electrical current is a measure of electron transfer, and can refer to flow of electrical charge, and/or the rate of charge flow through a circuit.

After formation of hybridization complexes, and while in contact with the redox solution, electrochemical reactions resulting in electron transfer to the electrode are detected by one or more methods described herein. In general, the detected electrochemical reactions are redox reactions in which the redox mediators in solution participate (i.e., are oxidized or reduced), and which redox reactions are catalyzed, and/or facilitated, and/or enhanced by the presence of the surface-attached nanoparticle, i.e., nanoparticles brought in proximity to the electrode surface by formation of a complex comprising capture probe and a target sequence.

In some embodiments of the present methods, an additional electrochemically active moiety is added to the redox solution in order to amplify the generated signal and thus enhance the detection limit of the catalytic electrochemical assay. In some embodiments, the electrochemically active moiety is a sacrificial electron donor. Suitable sacrificial electron donors include, but are not limited to, disodium ethylenediaminetetraacetic acid (EDTA), triethanolamine (TEOA), triethylamine (TEA), and tripropylamine (TPA). In some embodiments, the sacrificial electron donor is EDTA, which is dissolved in the redox electrolyte solution. Thus, in some embodiments, the oxidization of electron donors in solution (e.g., EDTA) is also detected as part of the electric signal in the electrode.

Nanoparticles can, in some embodiments, function as electron bridges in which electrons generated by the oxidation of redox mediators/donors in solution are transferred to the surface of the electrode via a hybridization complex comprising a nanoparticle. In one example, the surface-attached nanoparticle actually catalyzes a redox reaction. For example, a platinum nanoparticle can catalyze the oxidation of a redox solution comprising water. In such nanoparticle-catalyzed reactions, electrons can be transferred from the redox mediator in solution to the electrode surface. It is specifically noted, however, that this mechanism (i.e., electron transfer from redox mediator to electrode via a nanoparticle-comprising bridge) is not the only redox/electron transfer reaction that can be catalyzed and detected according to the present methods. Electrons may be transferred directly to the surface of the electrode (i.e., without traveling along the hybridization complex comprising a nanoparticle). In some embodiments, redox-active moieties attached to nanoparticles are oxidized by the electrode itself, while other redox-active moieties (e.g., electron donors) in solution reduce or re-reduce the nanoparticle-attached redox active moieties, thus generating electron transfer to the electrode.

Electron transfer to the electrode surface generates an electrical signal (i.e., current) in the electrode, which can be detected by any suitable method. Electrochemical and/or catalytic current is not generated in significant amounts by non-hybridized capture probes, because these capture probes are not also attached to a detection probe comprising a nanoparticle. Accordingly, detectable electric signal over background signal (where background signal is the signal generated by a “bare” electrode surface, i.e., an electrode surface not modified by nanoparticles) is indicative of nucleic acid hybridization, which can be correlated to the concentration of target sequence in a given sample. Stated another way, an increase in electron transfer to the electrode as compared to the amount of electron transfer to a bare, unmodified electrode indicates hybridization of the target sequence to the electrode.

In some embodiments, the generated electrical current is measured for each nanoparticle-modified electrode, and compared to a reference current obtained with the complex-free electrode. In some embodiments, a comparison is made between the electrode potential of the complex-free electrode and the potential of the modified electrode.

The detection of the electronic signal associated with one or more redox reactions permits the determination of the presence or absence of hybridized nucleic acid, and optionally the quantitation of the amount of nucleic acid in a sample. For example, determining the presence of hybridized nucleic acid can include (i) measuring the generation of current by the redox reaction and then (ii) comparing the generated current to the current generated by the complex-free electrode. Alternatively, determining the presence of hybridized nucleic acid can include (i) measuring the potential of the electrode supporting the redox reaction and then (ii) comparing the potential to the potential of the complex-free electrode, for example.

The detected signal can also be compared to a predetermined threshold or control. The control can be any appropriate control, such as a control under substantially the same conditions, except that no nucleic acids are present, or only non-target sequences are present.

In some embodiments, a competitive assay format is provided. Unlabeled sample target sequences compete with a predetermined amount of competitive, labeled sequences, for hybridizing to capture probes.

Measuring the electrical signal in the electrode can be carried out by any suitable approach. In some embodiments, the redox reaction is measured by measuring the electronic current associated with the occurrence of the redox reaction. The electrical signal associated with the redox reaction may be measured by providing a suitable apparatus in electronic communication with the electrode. A suitable apparatus will be capable of measuring the electronic signal that is generated, so as to provide a measurement of the redox reactions occurring in the redox solution and at the electrode. A positive current flow is indicative of nanoparticle attachment and thus hybridization complex formation. The current is detected and compared with an amount of current that is generated by a complex-free electrode (i.e., an electrode without attached nanoparticles). An increase in current in the modified electrode as compared to the complex-free electrode is indicative of nucleic acid hybridization at the electrode surface, and thus is indicative of the presence of target nucleic acid at the surface of the electrode.

Detection of generated electric signal in the form of current is carried out using one of any number of suitable means, including amperommetry, voltammetry, and capacitance and impedence detection techniques. Suitable techniques include, but are not limited to, electrogravimetry; coulometry (including controlled potential coulometry and constant current coulometry); voltammetry (cyclic voltammetry, pulse voltammetry (normal pulse voltammetry, square wave voltammetry, differential pulse voltammetry, Osteryoung square wave voltammetry, and coulostatic pulse techniques); stripping analysis (anodic stripping analysis, cathodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; and capacitance measurement.

A general definition for the term “voltammetry” is any electrochemical technique that involves controlling the potential of an electrode while simultaneously measuring the current flowing at that electrode. In voltammetry, current at a working electrode in solution is measured as a function of a potential waveform applied to the electrode. The resulting current-potential curve is called a voltammogram, and correct interpretation provides information about the reaction occurring at the surface of the electrode.

Voltammetry is usually performed by connecting an electrochemical potentiostat to an electrochemical cell. The cell contains a test solution and three electrodes. One of the three electrodes is the working electrode. The second electrode is a reference electrode, against which the potential of the working electrode is measured. The third electrode is called a counter electrode. The counter electrode is usually a piece of inert, conducting material such as platinum (Pt).

A device, generally a potentiostat, controls the potential of the working electrode. It is designed to work with a three electrode cell in a way which assures that all current will flow between counter and working electrodes, while controlling the potential of the working electrode with respect to the reference electrode. Special electronic circuitry within the potentiostat permits the working electrode potential to be controlled with respect to the reference electrode without any appreciable current flowing at the reference electrode. The simplest potentiostat has a means of setting the starting potential and the switching potential, a sweep rate adjustment, and outputs which monitor working electrode potential and current flow. These are connected to the X and Y axes of an X-Y recorder, respectively. Modern electrochemical systems are often “closed-box” systems that are controlled by a computer.

In a typical voltammetric experiment, oxidation or reduction of analytes occurs at the surface of a working electrode when the electrode is biased near the redox (Nernst potential) of a given analyte. At this potential, electron transfer takes place and a measurable change in current occurs whose magnitude is linearly proportional to the concentration of the given analyte in solution. Therefore, the magnitude of the current peak provides concentration information while the potential at which the current peak occurs identifies the analyte. Additional information such as the reaction type (reversible, quasi-reversible, and irreversible) and analyte mass-transport rates (diffusion coefficients) can also be obtained depending on the type of voltammetric experiment performed. Since most analytes have different redox potentials, voltammetry allows the measurement of multiple analytes in solution.

There are several variations of voltammetric measurements, and most of these are due to changes in the type of potential waveform (i.e., input/probe signal and/or shape of input/probe signal used to sweep the voltage range) used (e.g., cyclic, staircase, AC, squarewave, pulse, and differential pulse voltammetry) and/or the addition of a preconcentration step (stripping voltammetry). Consequently, the choice of technique determines how many characteristics of the redox reaction can be measured and how well a given characteristic can be measured.

The shape of a voltammogram gives information about the kinetics of electrode processes. The shape of the current peaks due to the “Faradaic processes” (this terminology is used to denote charge transfer processes) is determined by the concentration of the redox species at the electrode surface. In cyclic voltammetry the electrolyte solution is not stirred, and it is important that the system is at rest (i.e., no mechanical agitation) while the experimentation is performed. Under these conditions the surface concentration is governed by diffusion of the redox active species to the electrode surface.

For example, in cyclic voltammetry, a DC voltage sweep is done. In AC voltammetry, an AC signal is superimposed on to the voltage sweep. In square wave voltammetry, a square wave is superimposed on to the voltage sweep. Most preferably, the signal is recorded from each position (“address”) on an array (e.g., at one attachment point on an array).

In cyclic voltammetry, the voltage that is applied to the working electrode is an inverted triangle wave, so that the electrode potential becomes more negative linearly in time until it reaches a predetermined switching potential, at that point the potential of the working electrode is scanned to more positive potentials, again varying linearly in time. In cyclic voltammetry, the working electrode potential is swept back and forth across the formal potential of the analyte. Cyclic voltammograms trace the transfer of electrons during a redox reaction. The reaction begins at a certain potential (voltage). As the potential changes, it controls the point at which the redox reaction will take place. Repeated reduction and oxidation of the analyte causes alternating cathodic and anodic currents flow at the electrode.

Experimental results are usually plotted as a graph of current versus potential. The voltammogram exhibits two asymmetric peaks, one cathodic and the other anodic. The signal of primary interest to the artisan will be the height of the peak or peaks. The voltammogram can provide information about both the oxidation and reduction reaction which includes the thermodynamics of the redox processes, the kinetics of heterogeneous electron transfer reactions, analyte identification and quantitation, and analyte diffusion coefficients.

Cyclic voltammetry (CV) is advantageously used to study the electroactivity of compounds, particularly biological molecules. In particular, it is well suited to probe-coupled chemical reactions, particularly to determine mechanisms and rates of oxidation/reduction reactions. Moreover, cyclic voltammetry can be used to study electrode surfaces and the reactions that take place thereon.

Stripping voltammetry techniques such as anodic stripping voltammetry, cathodic stripping voltammetry, potentiometric stripping analysis and adsorptive stripping voltammetry can also be used with the present methods.

In addition to voltammetry, other methods such as chronoamperometry can be used. In chronoamperometry, the working electrode potential is suddenly stepped from an initial potential to a final potential, and the step usually crosses the formal potential of the analyte. The solution is not stirred. The initial potential is chosen so that no current flows (i.e., the electrode is held at a potential that neither oxidizes or reduces the predominant form of the analyte). Then, the potential is stepped to a potential that either oxidizes or reduces the analyte, and a current begins to flow at the electrode. This current is quite large at first, but it rapidly decays as the analyte near the electrode is consumed, and a transient signal is observed.

In some embodiments, cyclic voltammetry is used to measure the current in the electrode, and the apparatus used comprises a plurality of electrodes, including the electrode upon which the hybridization reactions are carried out (i.e., the working electrode), at least one counter-electrode and optionally a reference electrode, and an electrolyte solution in contact with the plurality of microelectrodes, counter electrode and reference electrode. The working electrode may, as set forth above, be supported by a solid substrate. The solid substrate may comprise one working electrode, or a plurality of working electrodes. In some embodiments, a solid substrate may comprise a plurality or microelectrodes on its surface.

In some embodiments, the hybridization reactions set forth above are carried out in a reaction chamber located within a suitable electrochemical cell. Following hybridization of a target probe to an array of capture probes on an electrode surface, and hybridization of detection probes to any captured target sequences on the electrode, the electrodes are thoroughly rinsed in an excess volume of buffer, generally at room temperature. After washing, a suitable volume of a redox solution, as set forth above, is added to the reaction chamber, and each working electrode is interrogated by conventional cyclic voltammetry to detect a redox signal. The reaction chamber may optionally comprise at least two compartments, the working electrode compartment and the counter electrode compartment. The counter electrode compartment can be separated from the working electrode compartment by means of a gas permeable separator, which allows passage of a buffer solution and gases between the compartments, but does not permit passage of the reactants, e.g., the redox mediator. Suitable gas permeable separators can be made, for example, from glass, dialysis membranes, and Teflon-based materials, such as Nafion™.

The counter electrode can be made of any suitable material that is noncorrosive in the electrochemical cell and reaction solutions utilized. A preferred counter electrode is made of a material that is capable of supplying oxygen or hydrogen to the reaction vessel during the reaction, such as a platinum group metal, a metal oxide, and/or a carbon-based material. Particular counter electrode materials include palladium; ruthenium; platinum as wires, sheets or thin films; ruthenium oxide; glassy carbon; reticulated carbon; titanium dioxide; and mixed metal oxides.

Any suitable reference electrode can be used, such as a Ag/AgCl electrode, a calomel reference electrode or a normal hydrogen electrode.

J. Uses and Advantages of Methods

In a broad aspect, the methods described herein relate to electrochemical systems for detecting specific target sequences by using nanoparticles and target-specific probes. These methods have applications in regard to detecting identified nucleic acids in complex mixtures, and are particularly useful for assaying virtually any species so long as an identifiable sequence can be determined. Diagnostic assays, such as for aberrant chromosomal variations, cancers and genetic abnormalities are facilitated by methods described herein to the extent that targeted nucleic acid sequences or segments can be selectively probed employing the described methods.

The described methods can be employed to detect hybridization on an array and can be employed, for example, in sequencing, in mutational analysis (single nucleotide polymorphisms and other variations in a population), and for monitoring gene expression by analysis of the level of expression of messenger RNA extracted from a cell. Thus, examples of the uses of the methods of detecting nucleic acids include the diagnosis and/or monitoring of viral and bacterial diseases, inherited disorders, and cancers where genes are associated with the development of cancer; in forensics; in DNA sequencing; for paternity testing; for cell line authentication; for monitoring gene therapy; and for many other purposes.

Moreover, methods described herein can be employed to monitor hybridization events in a variety of different systems and models. As described more fully below, the present methods are particularly useful in the monitoring of gene expression, the detection of spontaneous or engineered mutations and in the design of probes.

In some embodiments, the present methods can be used to monitor gene expression. In some embodiments, single stranded DNA derived from a gene of interest is used as capture probe. Unexpressed sequences of DNA (for example introns) can be removed before the samples are attached to the support. In this application, it can be desirable to employ cDNA as a probe sequence. Control samples of unrelated single-stranded DNA can also be included to serve as an internal validation of the experiment.

Total mRNA is then isolated from an expression system using standard techniques, which mRNA serves as the target nucleic acid. Target mRNA can optionally be fragmented for ease of handling. The target mRNA is hybridized to the capture probe as described herein. A detection probe comprising a nanoparticle-oligonucleotide complex is then contacted with the support-bound target. In some embodiments of the method, conditions of high stringency are maintained, although these conditions can be varied with the needs and goals of the experiment. The electrode can be washed to remove any unhybridized sample.

Electrons produced by one or more redox reactions (e.g., electrons generated by the redox mediator in solution) are transferred to the electrode surface, and the resulting current flow is detected in the electrode. Gene expression can be determined by comparing duplex formation by the control sequences to duplex formation observed in the target samples. Appropriate mathematical descriptions and treatments of the observed duplex formation can indicate the degree of observed hybridization and consequently the degree of gene expression.

In some embodiments, the present methods can also be employed in the detection of mutations in a nucleic acid sequence. Such mutations can be engineered or spontaneous. For example, the present methods can be useful in determining whether an engineered mutation is present in a nucleic acid sequence, or for determining if a nucleic acid sequence contains deviations from its wild type sequence.

In these embodiments, single-stranded oligonucleotide probes are initially prepared. The probes can be known or suspected to contain a mutation(s) to be identified. Capture probe samples are attached to the support using methods described herein. Nucleic acid target sequences to be screened for the mutation are isolated from an expression system, and single stranded target sequences are prepared. If desired, large quantities of sample can be conveniently prepared using established amplification methods, as set forth above. Probe sequences are bound to a nanoparticle to form a detection probe, which is contacted with the capture probe-target hybridization complexes. Those probe sequences containing the mutation of interest will hybridize with the target sequence to form detectable complexes. Unbound target sequences can be removed by washing. The support, which can comprise any formed duplexes, is then interrogated for any electric current generated in the presence of a redox solution, and the resulting current detected. In these embodiments, a mutation can be located on either a target sequence or on a probe sequence, the selection of which can be made during experimental design.

In some embodiments, the present methods can be employed in designing nucleic acid probes. The ability to detect hybridization events permits a researcher to optimize a probe for the needs of a given experiment. For example, a probe can be designed that will accommodate a degree of polymorphism in a target sample. Such a probe can be useful for screening for genes or sequences known to exhibit polymorphisms. Using the present invention, it is possible to design a probe that will tolerate a degree of uncomplementarity in the sequence.

Additionally, the present methods can be used to screen for duplex formation between a target sequence and a polymorphic probe; that is, a probe that has one or more mutations from the wild type sequence. By varying the number of bases different from the wild type sequence, a desired degree of promiscuity in a probe can be obtained.

In this context, the present methods can be useful for detecting hybrid formation in sequential rounds of probe design. For example, if a designed probe binds only to the wild type sequence, no polymorphism is recognized; if the probe binds to sequences unrelated to the target sequence, the probe is not useful to identify the sequence of interest. By monitoring hybrid formation at each round of optimization, the present invention can be useful for nucleic acid probe design.

### EXAMPLES

The following Examples have been included to illustrate some modes of the invention. Certain aspects of the following Examples are described in terms of techniques and procedures found or contemplated by the present inventors to work well in the practice of the invention. In light of the present disclosure and the general level of skill in the art, those of skill will appreciate that the following Examples are intended to be exemplary only and that numerous changes, modifications and alterations can be employed without departing from the spirit and scope of the invention.

### Example 1

### Modification of Indium Tin Oxide (ITO) with Single-Stranded DNA

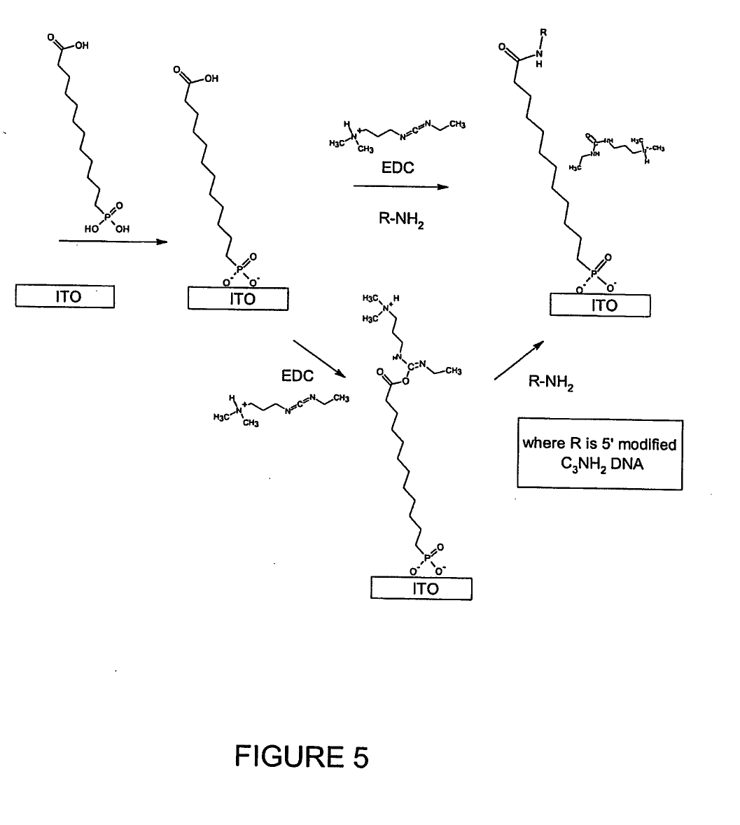

FIG. 5 outlines one strategy employed in the modification of indium tin oxide (ITO) with single-stranded DNA (ssDNA). Initially, a monolayer of 12-phosphonododecanoic acid (10 mM in 50/50 DMSO/18 MD cm H₂O for 16 hours) was formed on the ITO surface (cleaned 20 minutes with UV/O₃ (UVO-cleaner (UVO-60), model number 42, Jelight Company, Inc.)). The carboxylic acid of 12-phosphonododecanoic acid was then activated by 1-ethyl-3-(3-dimethylaminopropyl)carbodiimide hydrochloride (EDC) to form an O-acylisourea intermediate. See, e.g., S. H. Brewer et al., Langmuir (2002) 18, 6857-6865; B. L. Frey and R. M. Corn, Analytical Chemistry (1996) 68, 3187-3193; M. Burgener et al., Bioconjugate Chemistry (2000) 11, 749-754; K. Kerman et al., Analytica Chimica Acta (2002) 462, 39-47; E. Huang et al., Langmuir (2000) 16, 3272-3280; and G. T. Hermanson, Bioconjugate Techniques (1996) (Academic Press: San Diego).

This activated carboxylic acid group is attacked by the primary amine (acting as a nucleophile) of a 5′-modified C₃H₂ ssDNA strand to form an amide bond between the monolayer of 12-phosphonododecanoic acid and the 5′ modified C₃H₂ ssDNA. The coupling conditions were 1 μM 5′-modified C₃H₂ ssDNA and 200 mM EDC for 4 hours in a 0.1 M MES (2-(N-morpholino)ethane sulfonic acid) buffer at pH 5 with 0.25M NaCl.

Complementary 18-base pair single-stranded DNA sequences were attached to ITO and 10 nm diameter gold nanoparticles according to the foregoing methods.

### Example 2

### X-Ray Photoelectron Spectroscopy Characterization of ITO Electrode Surfaces Modified by Single Stranded DNA and Gold Nanoparticles

X-ray photoelectron spectroscopy (XPS) spectra were recorded on a Riber LAS 2000 Surface Analysis System equipped with a cylindrical mirror analyzer (CMA) and a MAC2 analyzer with Mg Kα X-rays (model CX 700 (Riber source) (hν=1253.6 eV). The elemental scans had a resolution of 1.0 eV and were the result of 5 scans. XPS spectra were smoothed using a 9 point (second order) Savitzky-Golay algorithm, baseline corrected and the peaks were fitted using Gaussian line shapes.

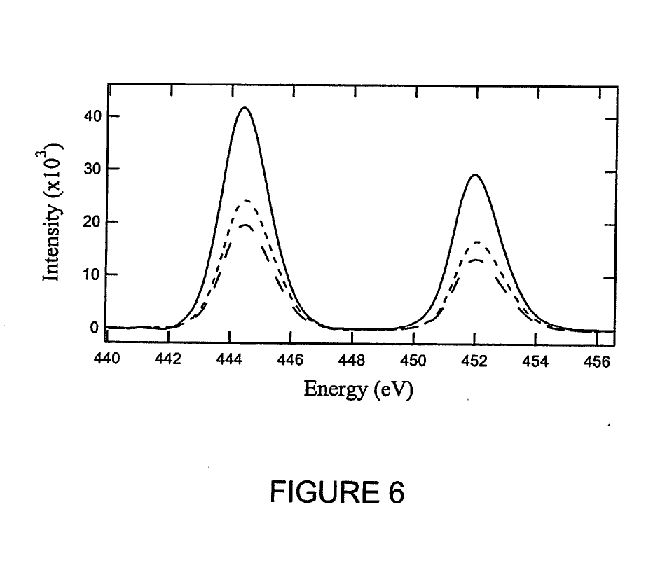

The results of these experiments is shown in FIGS. 6, 7, 8 and 9.

FIG. 6 is the x-ray photoelectron spectra (XPS) of ln 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

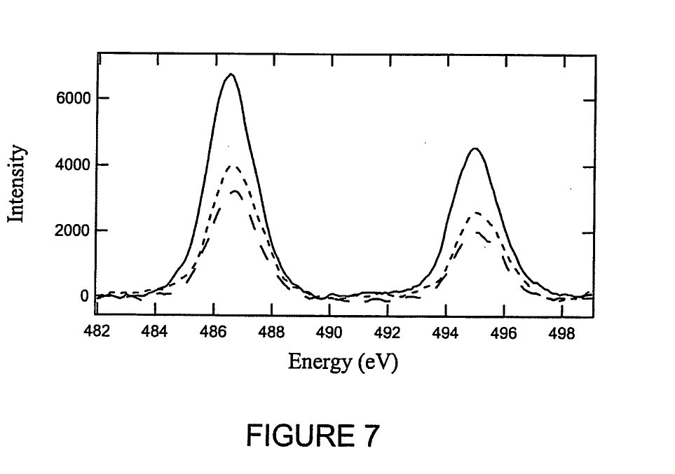

FIG. 7 is the XPS spectra of Sn 3d_{5/2,3/2} for bare ITO (solid), ITO modified with a monolayer of 12-phosphonododecanoic acid (short dash) and ITO modified with ssDNA coupled through a monolayer of 12-phosphonododecanoic acid (long dash).

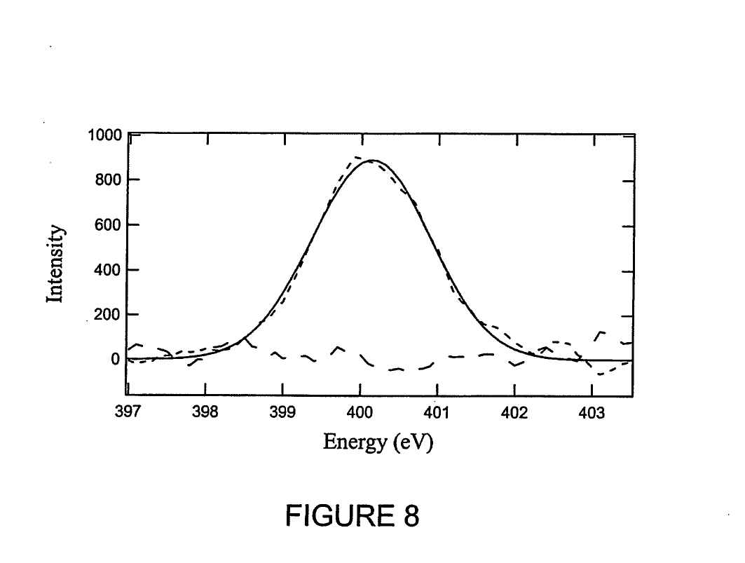

FIG. 8 is the XPS N 1s spectra of ITO modified with a monolayer of 12-phosphonododecanoic acid (long dash) and ITO modified with ssDNA coupled through a monolayer of 12-phosphonododecanoic acid (short dash) fitted to a Gaussian line shape (solid).

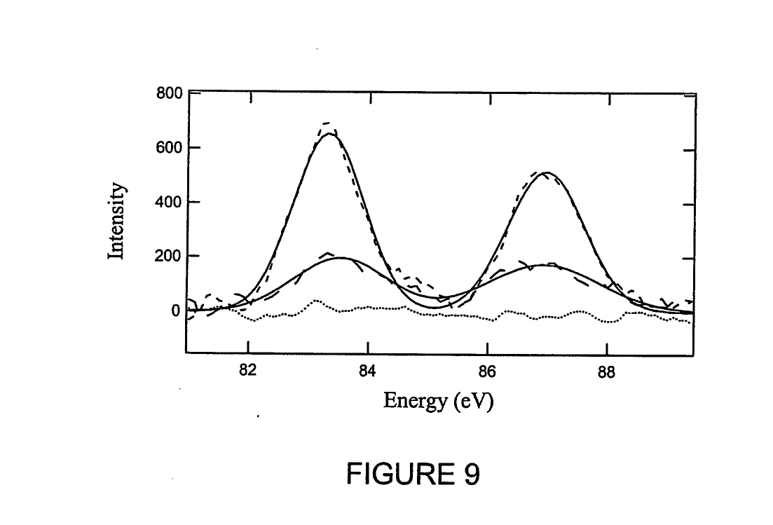

FIG. 9 is the XPS Au 4f_{7/2,5/2} spectra of ITO modified with ssDNA coupled through a monolayer of 12-phosphonododecanoic acid (dotted line) exposed to the complementary (short dash) or non-complementary (long dash) ssDNA labeled with a 10 nm gold nanoparticle (1 nM) fitted to two Gaussian line shapes (solid).

### Example 3

### Signal Enhancement from Surface-Bound Gold Nanoparticles in Presence of EDTA

A large enhancement in the signal from surface bound gold nanoparticles in the presence of EDTA. Notably, the signal enhancement is observed in the absence of light or other radiation excitation. It was determined that the enhancement in electrochemical signal is not due to stripping and is reversible, and further determined that the enhancement phenomenon is the result of gold nanoparticle catalysis of EDTA oxidation on ITO electrodes.

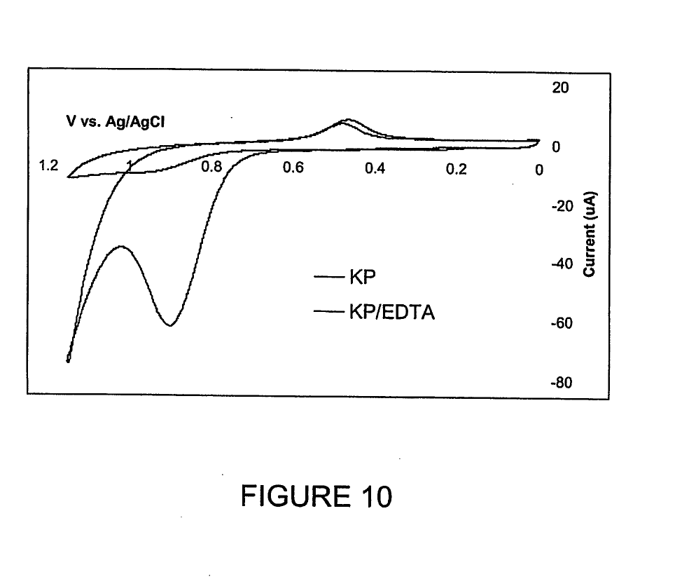

FIG. 10 is a graph comparing the cyclic voltammogram trace of gold nanoparticles hybridized onto ITO electrodes when the electrode solution comprises an electrolyte solution without EDTA (KP buffer only, upper trace/small current peak observed) and with EDTA (KP buffer/EDTA, lower trace/large current peak observed). In the absence of EDTA, redox waves associated with gold oxide formation and re-reduction are observed. When EDTA is added to the redox solution, the gold oxide wave increases in magnitude. EDTA oxidation is slow on bare ITO, and thus is not observed. When gold nanoparticles are present, however, EDTA is oxidized rapidly and a large current is observed.

### Example 4

Detection of Gold Nanoparticles Bound to ITO Through Aminosilane

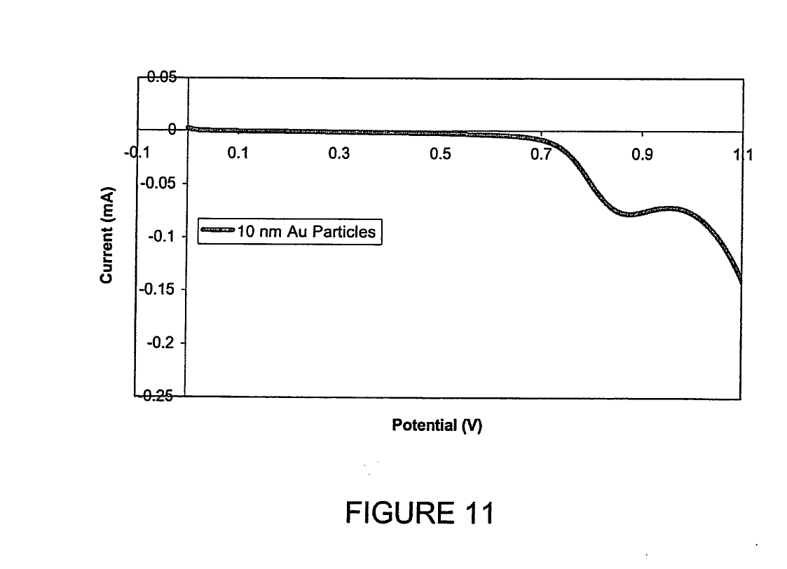

FIG. 11 is a graph of current as a function of sweeping potential illustrating the detection of gold particles bound to indium tin oxide electrodes using aminosilane attachment chemistry as described herein. In the experiments illustrated in FIG. 11, 1 pmole of 10 nm gold particles were capped with citrate and attached to the surface of an ITO electrode using the aminosilane attachment chemistry described in K. C. Grabar et al., J. Am. Chem. Soc. (1996) 118, 1148. Electrochemistry was carried out in a solution of 100 mM KP buffer/50 mM EDTA, pH. 7.3, and with a potential sweep rate of 100 mV/s. A detectable peak is observed at about 0.9 V.

### Example 5

### Catalytic Nanoparticle Electrochemistry of Nucleic Acid Detection

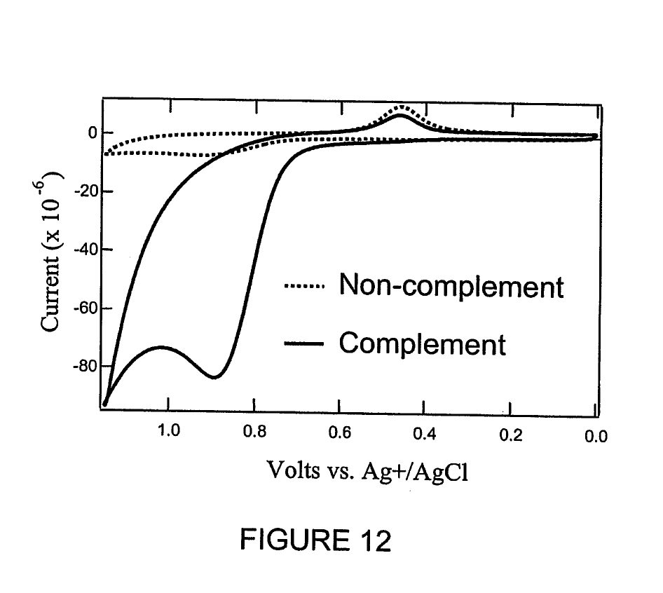

FIG. 12 is a comparison of cyclic voltammetry traces between gold particles bound to indium tin oxide electrodes when complementary single stranded DNA is attached to each (solid line) and when non-complementary single stranded DNA is attached to each (broken line). The cyclic voltammagram obtained in 100 mM FeCl₂; 100 mV/s scan rate; Indium-tin oxide substrate cleaned for 15 minutes by UV-ozonolysis; hybridization for 19 hours at 37° C. while gently stirring the solution; 10 picomolar target oligonucleotide in solution labeled with 10 nm gold particles stabilized with BSPP.

### Example 6

### Electrochemical Detection Limits

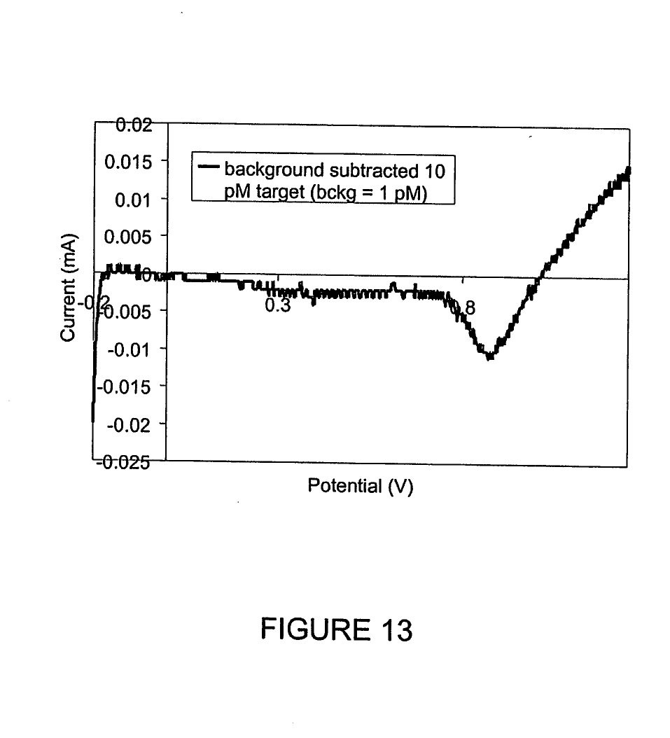

FIG. 13 is an illustration of the limits of detection of methods of the present invention. The present electrochemical methods are able to detect (i.e., distinguish over background) hybridization of nucleic acids at electrode surfaces in concentrations as low as about 10 μM. The cyclic voltammagram obtained in 100 mM FeCl₂; 100 mV/s scan rate; Indium-tin oxide substrate cleaned for 15 minutes by UV-ozonolysis; hybridization for 19 hours at 37° C. while gently stirring the solution; 10 picomolar target oligonucleotide in solution labeled with 10 nm gold particles stabilized with BSPP.

It will be understood that various details of the presently disclosed subject matter can be changed without departing from the scope of the presently disclosed subject matter. Furthermore, the foregoing description is for the purpose of illustration only, and not for the purpose of limitation.

1. A method of detecting a target nucleic acid, comprising:
providing a hybridization complex comprising (a) a capture probe that is attached to an electrode surface directly or via an attachment linker; (b) a target nucleic acid that is hybridized to the capture probe; and (c) at least one nanoparticle attached to the target nucleic acid;
removing unhybridized nucleic acid;
contacting the electrode with a redox solution comprising a redox mediator and an electrolyte, such that the complex is in contact with the redox solution; and
detecting a non-photogenerated electrical signal in the electrode, whereby detection of an increased non-photogenerated electrical signal relative to a signal that would be detected in the absence of said target nucleic acid or said nanoparticle indicates the presence or amount of target nucleic acid hybridized to the electrode.

2. The method of claim 1, wherein providing the hybridization complex comprises:
hybridizing a target nucleic acid to at least one capture probe to form a capture probe-target nucleic acid complex; and
hybridizing a detection probe to the capture probe-target nucleic acid complex to form the hybridization complex, wherein the detection probe comprises the nanoparticle.

3. The method of claim 1, wherein the target nucleic acid comprises RNA.

4. The method of claim 1, wherein the target nucleic acid comprises cDNA.

5. The method of claim 1, wherein the target nucleic acid is present in a biological sample.

6. The method of claim 1, wherein the electrode comprises a conducting material comprising one or more of metals and metal oxides.

7. The method according to claim 1, wherein the electrode comprises indium tin oxide.

8. The method according to claim 1, wherein the electrode is formed on a non-conducting solid substrate.

9. The method according to claim 1, wherein the nanoparticle comprises one or more of the group consisting of metals and metal oxides.

10. The method according to claim 9, wherein the nanoparticle comprises a metal comprising one or more of gold, silver, platinum and palladium.

11. The method according to claim 1, wherein the nanoparticle comprises gold.

12. The method according to claim 1, wherein the nanoparticle comprises silver.

13. The method according to claim 1, wherein the nanoparticle has a diameter from about 10 to about 20 nanometers.

14. (canceled)

15. The method according to claim 1, where the nanoparticle is attached to the target nucleic acid by one of the group consisting of a binding pair and complementary nucleic acids.

16. The method according to claim 1, where the nanoparticle is attached to the target nucleic acid by one of the group consisting of primer extension and ligation of a nanoparticle-labeled nucleic acid.

17. The method of claim 1, wherein the complex comprises a detection probe.

18. The method of claim 17, wherein the detection probe is attached to the target nucleic acid before, during, or after the target nucleic acid hybridizes to the capture probe.

19. The method of claim 1, comprising the sequential steps of hybridizing the target nucleic acid to the capture probe; and then reacting the hybrid with a detection probe.

20. The method according to claim 2, wherein the detection probe further comprises an oligonucleotide attached to the nanoparticle.

21. The method according to claim 20, wherein the capture probe is complementary to a first target domain of the target nucleic acid, and the oligonucleotide of the detection probe is complementary to a second target domain of the target nucleic acid.

22. The method according to claim 2, wherein the detection probe further comprises one partner of a ligand-binding pair, and the target nucleic acid comprises the other partner of a ligand-binding pair.

23. The method according to claim 22, wherein one partner of the ligand-binding pair is streptavidin, and the other partner of the ligand binding pair is biotin.

24. The method according to claim 22, wherein the target nucleic acid comprises biotin.

25. The method according to claim 24, wherein the biotin has been incorporated into the target nucleic acid during nucleic acid amplification.

26. The method according to claim 22, wherein the detection probe comprises streptavidin.

27. The method according to claim 1, wherein the redox mediator comprises EDTA.

28. The method according to claim 1, wherein the redox mediator comprises ferrocene.

29. The method according to claim 1, wherein the electrical signal is electrical current, and the detecting step is carried out by cyclic voltammetry.

30. The method according to claim 1, wherein the detecting step is carried out by chronoamperometry.

31. The method according to claim 1, wherein a plurality of different capture probes is attached to the electrode in an array.

32. The method according to claim 1, wherein the redox solution further comprises a sacrificial electron donor.

33. The method according to claim 32, wherein the sacrificial electron donor comprises EDTA.

34. The method according to claim 1, wherein the target nucleic acid is selected from the group consisting of an mRNA sequence derived from a biological sample and a cDNA sequence derived from a biological sample.

35. The method according to claim 34, wherein an indication of hybridization complex formation is indicative of gene expression or a gene expression level.

36. The method according to claim 1, wherein the capture probe comprises a nucleic acid from a gene of interest.

37. The method according to claim 1, wherein a redox reaction catalyzed by the nanoparticle generates electron transfer to the electrode, resulting in a detectable electrical signal in the electrode.

38. The method according to claim 1, wherein:
the nanoparticle comprises platinum;
the redox solution comprises water;
the nanoparticle oxidizes the water, wherein the oxidation generates electrons; and
the electrons are transferred to the electrode, resulting in a detectable electrical signal in the electrode.

39. The method of claim 1, wherein the nanoparticle is free of a photochemically active moiety.

40. A method of detecting a target nucleic acid, comprising:
providing a hybridization complex comprising (a) a capture probe that is attached to an electrode surface directly or via an attachment linker; (b) a target nucleic acid that is hybridized to the capture probe; and (c) at least one nanoparticle attached to the target nucleic acid;
removing unhybridized nucleic acid;
contacting the electrode with a redox solution comprising a redox mediator and an electrolyte, such that the complex is in contact with the redox solution; and
detecting an electrical signal in the electrode, whereby detection of an increased electrical signal relative to a signal that would be detected in the absence of said target nucleic acid or said nanoparticle indicates the presence or amount of target nucleic acid hybridized to the electrode, wherein the method is free of exposing a photoelectrochemically active moiety to a laser.
